(19) United States
(12) Patent Application Publication
(10) Pub. No.: US 2026/0290529 A1
(43) Pub. Date: Sep. 24, 2026

(54) Identification of Medical Intervention Related Adverse Events from Clinical Notes

(71) Applicants: The Regents of the University of California, Oakland, CA (US); The United States of America, as represented by the Secretary, Department of Health and Human, Silver Spring, MD (US)

(72) Inventors: Vivek Rudrapatna, San Francisco, CA (US); Rebecca Hernandorena, San Francisco, CA (US); Mahalakshmi Parakala, San Francisco, CA (US); Madhumita Sushil, San Francisco, CA (US); Ludwig Dana, San Francisco, CA (US); Balu Bhasuran, San Francisco, CA (US); Anna Silverman, San Francisco, CA (US); James Buchanan, San Francisco, CA (US)

(21) Appl. No.: 19/475,721

(22) PCT Filed: May 8, 2024
(86) PCT No.: PCT/US2024/028389

§ 371 Date: Oct. 15, 2025

Related U.S. Application Data
(60) Provisional application No. 63/465,382, filed on May 10, 2023.

(51) Int. Cl. G16H 15/00; G16H 10/60

(52) U.S. Cl. CPC G16H 15/00; G16H 10/60

(57) ABSTRACT

Methods of identifying medical intervention related adverse events (AEs) from electronic health records (EHR) data are provided. Aspects of the methods include: obtaining a plurality of health records comprising clinical notes regarding individuals associated with the medical intervention; preprocessing the plurality of clinical notes to include medical intervention and adverse event labels; annotating a first subset of the preprocessed clinical notes to indicate if each labeled adverse event is associated with the medical intervention; training a machine learning model to identify adverse events associated with the medical intervention using the annotated clinical notes; and applying the trained machine learning model to a second subset of the preprocessed clinical notes to identify adverse events associated with the medical intervention. Aspects of the present invention further include methods of identifying contexts of AEs indicative of the nature or severity of AEs of interest. Also provided are systems for performing the methods described herein as well as non-transitory computer readable storage media and computer products.

### STATEMENT REGARDING FEDERALLY SPONSORED RESEARCH

This invention was made with government support under K99 LM014099 awarded by the National Institutes of Health, and U01 FD005978 awarded by the Food and Drug Administration. The government has certain rights in the invention.

### CROSS-REFERENCE TO RELATED APPLICATION

Pursuant to 35 U.S.C. § 119(e), this application claims priority to the filing date of United States Provisional Patent Application Ser. No. 63,465,382 filed May 10, 2023, the disclosure of which is herein incorporated by reference in its entirety.

### INTRODUCTION

The accurate detection of treatment-related adverse events (AEs) is critical to ensure that clinicians and patients can make well-informed treatment decisions that balance risks with benefits. This is particularly true of steroid-sparing immunosuppressants which are commonly needed long-term for the treatment of inflammatory bowel diseases (IBD). The last few decades have seen a significant expansion in United States Food and Drug Administration (FDA)-approved therapies for IBD. In the current era of IBD treatment with numerous agents available, now more than ever, additional information to support therapy selection is needed, including safety data.

Existing approaches for AE surveillance typically require a combination of prospective registry studies, spontaneous post-marketing reporting, and analyses of the structured data from claims and electronic health records databases. These approaches have provided important data on the safety of medications but are limited by expense, small numbers, and suboptimal sensitivity and specificity. Clinical notes are a rich source of AE data because treating clinicians often document the reasons for treatment-discontinuation; however, these have been underutilized for surveillance due to methodological limitations in effective text mining. Indeed, many aspects make the task of AE detection from clinical notes particularly difficult. These aspects include, e.g., the length of typical clinical notes, the need to infer relationships between medications and documented AEs, the need to encode AEs in a standardized way, assumed knowledge of the specialty the clinical notes are written for including domain-specific abbreviations and terminology, and the inherent vagueness in the documentation of clinical notes, just to name a few.

### SUMMARY

Thus, there is a need for improved and useful methods and systems for accurately interpreting medical language and inferring relationships between medical interventions and adverse events. This invention provides such new and useful methods and systems, addressing the limitations mentioned above. To accomplish this, the invention leverages electronic health record data, both rule-based approaches and supervised machine learning approaches, and human domain expertise as well as recent advances in machine learning techniques and natural language processing tools to automatically perform predictive tasks, infer relationships, and identify features using structured healthcare data and unstructured healthcare data such as clinical notes. The methods and systems of the invention, e.g., as described in greater detail below, find use in a variety of applications where it is desirable to accurately and cost effectively monitor the safety of various medical interventions such as the safety of drugs and medical devices.

Methods of identifying medical intervention related adverse events (AEs) from electronic health records (EHR) data are provided. Aspects of the methods include: obtaining a plurality of health records including clinical notes regarding individuals associated with the medical intervention; preprocessing the plurality of clinical notes to include medical intervention and adverse event labels; annotating a first subset of the preprocessed clinical notes to indicate if each labeled adverse event is associated with the medical intervention; training a machine learning model to identify adverse events associated with the medical intervention using the annotated clinical notes; and applying the trained machine learning model to a second subset of the preprocessed clinical notes to identify adverse events associated with the medical intervention. Aspects of the present invention further include methods of identifying contexts of AEs indicative of the nature or severity of AEs of interest. Also provided are systems for performing the methods described herein as well as non-transitory computer readable storage media and computer products.

### BRIEF DESCRIPTION OF THE FIGURES

FIG. 1 illustrates prediction tasks performed by a machine learning model in accordance with an embodiment of the invention.

FIG. 2 provides a flow diagram depicting a method of identifying medical intervention related AEs from EHR data in accordance with an embodiment of the invention.

FIG. 3 provides an overview of a method of identifying medical intervention related AEs from EHR data in accordance with an embodiment of the invention.

FIGS. 4A to 4Y provide an output table of medical intervention related AEs generated from EHR data using a machine learning model in accordance with an embodiment of the invention.

FIG. 5 depicts a network graph of serious AEs (SAEs) by medication class produced using a machine learning model in accordance with an embodiment of the invention.

### DETAILED DESCRIPTION

Methods of identifying medical intervention related AEs from clinical notes are provided. Aspects of the methods include: obtaining a plurality of health records including clinical notes regarding individuals associated with the medical intervention; preprocessing the plurality of clinical notes to include medical intervention and adverse event labels; annotating a first subset of the preprocessed clinical notes to indicate if each labeled adverse event is associated with the medical intervention; training a machine learning model to identify adverse events associated with the medical intervention using the annotated clinical notes; and applying the trained machine learning model to a second subset of the preprocessed clinical notes to identify adverse events associated with the medical intervention. Aspects of the present invention further include methods of identifying contexts of AEs indicative of the nature or severity of AEs of interest. Also provided are systems for performing the methods described herein as well as non-transitory computer readable storage media and computer products.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates, which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods

As summarized above, methods of identifying medical intervention related AEs from clinical notes are provided. Aspects of the methods include: obtaining a plurality of health records including clinical notes regarding individuals associated with the medical intervention; preprocessing the plurality of clinical notes to include medical intervention and adverse event labels; annotating a first subset of the preprocessed clinical notes to indicate if each labeled adverse event is associated with the medical intervention; training a machine learning model to identify adverse events associated with the medical intervention using the annotated clinical notes; and applying the trained machine learning model to a second subset of the preprocessed clinical notes to identify adverse events associated with the medical intervention.

### Obtaining Electronic Health Records

As described above, embodiments of the methods include obtaining a plurality of health records including clinical notes regarding individuals associated with the medical intervention. By obtain is meant to make the plurality of health records accessible or available for the subsequent steps of the methods (e.g., available for preprocessing, annotating, training, etc.). The plurality of health records may be obtained through any available means, and from any available source. In some embodiments, the health records may include electronic health records (EHRs). By electronic health record is meant health information about an individual existing in a digital form. The EHR data may include structured data (i.e., data stored in a predefined structured format, e.g., for ease of sorting, searching and analysis) and unstructured data (e.g., clinical notes). In some embodiments, the EHR data may be obtained by gaining access to a database storing a corpus of EHR data including clinical notes regarding individuals associated with the medical intervention.

In some embodiments, the plurality of health records (e.g., EHR data) obtained may be de-identified to redact patient-specific information (e.g., names, geographical subdivisions smaller than state level, contact information, social security numbers, identifying biometric information, etc.) and protected health information (PHI). In some cases, PHI data may be machine redacted in order to, e.g., produce PHI-redacted clinical notes regarding individuals associated with the medical intervention. In these instances, certain identifying or protected information may be randomized. For example, the true dates of each clinical note or medical event (e.g., hospitalization) may be randomly shifted backwards in time by up to a calendar year.

In some embodiments, the EHR data (e.g., digitally accessible deidentified patient records) may be obtained by transmitting the data from a database storing a corpus of EHR data. Transmitting can include any manner of sending, passing, or conveying the EHR data from the database to a means for performing a subsequent step or steps of the methods (e.g., a processor, computer program or application, lines of computer code, etc.). In some instances, the EHR data may be obtained, at least in part, by converting the data to a form compatible with a subsequent step or steps of the methods. In some embodiments, the EHR data may be converted from a format difficult for machines to interpret to a format in a standard computer language that can be read automatically by a machine. In some cases, optical character recognition (OCR) software may be used to convert EHR data to a form compatible with a subsequent step or steps of the methods. For example, in cases where EHR data is stored in an image format (e.g., a PDF or JPEG format), the EHR data may be converted to a JSON format, an XML format, a CSV format, a CSON format, an HTML format, etc. In these cases, organizational or categorical information structuring or classifying the EHR data may be manually entered. For example, clinical notes, or sections thereof, may be categorized using dates, diagnosis codes (such as, e.g., diagnosis codes associated with hospitalizations), medical departments or units, section headers (such as, e.g., the header for a history of present illness section), etc. In some cases, organizational or categorical information may be automatically identified from structured EHR data and used to structure or classify clinical notes, or sections thereof, using, e.g., lines of computer code and rules-based approaches. In some instances, the EHR data may be obtained by scanning or imaging a plurality of health records existing in hard copy form, followed optionally by conversion of the resulting image files in any of the manners discussed above.

In embodiments where EHR data is obtained from a database storing a corpus of EHR data, the database may be aggregated or generated by any entity, including, but not limited to, a medical center, a nonprofit organization, an insurance provider, a government organization, an academic institution, a pharmaceutical or medical corporation, etc. The plurality of health records may include the EHR data of individuals of any demographic or cohort. For example, the EHR data may regard individuals of any sex, gender, age, ethnicity, or race. In some cases, the plurality of health records may include the EHR data of individuals associated with a population or cohort of interest. By population or cohort of interest is meant a group of people banded together or treated as a group, such as a specific demographic of individuals. For example, the cohort of interest may be individuals experiencing or affected by (e.g., at risk for) a specific disease or condition. In these cases, the disease or condition may be any disease or condition that impairs or affects the normal functioning of the body. In some instances, the disease or condition may be, e.g., an infectious disease, deficiency disease, hereditary disease, or physiological disease.

In embodiments where the disease or condition is an infectious disease, the infectious disease may be, e.g., a bacterial disease or infection (such as, e.g., syphilis, pneumonia, tetanus, and/or tuberculosis), a viral disease or infection (such as, e.g., chickenpox, measles, herpes, the common cold, or COVID-19), a fungal disease or infection (such as, e.g., ringworm infection, athlete's foot, or yeast infections), or a parasite or parasitic disease (such as, e.g., malaria).

In embodiments where the disease or condition is a deficiency disease, the deficiency disease may be, e.g., malnutrition, scurvy, rickets, osteoporosis, or a birth defect. In embodiments where the disease or condition is a hereditary disease, the hereditary disease may be, e.g., cystic fibrosis, Huntington's Disease, sickle cell anemia, a birth defect, etc. In some cases, the disease or condition may be affected by, but not unilaterally caused by, genetics or may be a polygenic disease. In these instances, the disease or condition may be caused by a combination of genetic and environmental factors and may be asthma, an autoimmune disease such as multiple sclerosis, cancer, ciliopathy, cleft palate, diabetes, heart disease, hypertension, inflammatory bowel disease, an intellectual disability, a mood disorder, obesity, refractive error, infertility, schizophrenia, or any number of a variety of mental disorders. In embodiments where the disease or condition is a physiological disease, the physiological disease may be, e.g., diabetes, cancer, hypertension, or heart disease. In some cases, the disease or condition may include any disease or condition caused by environmental factors, behavior, or diet.

In some cases, the disease or condition may psychological disease or condition such as, e.g., an anxiety disorder, depression, bipolar disorder, post-traumatic stress disorder (PTSD), schizophrenia, an eating disorder, a disruptive behavior and/or dissocial disorder, or a neurodevelopmental disorder. In some instances, the disease or condition may be hypothermia, hyperthermia, or toxin exposure or may result from exposure to prolonged or extreme hot or cold temperatures. In some cases, the disease or condition may result from an injury or may affect mobility. For example, the disease or condition may include, but is not limited to, a burn, cuts or scrapes, internal bleeding, traumatic injuries, arthritis, tendonitis, a tendon or myotendinous tear, a hernia, old age, paralysis, chronic health problems such as, e.g., chronic pain or chronic problems associated with bad posture, etc.

In some instances, the cohort of interest may include individuals diagnosed with a specific disease or condition. In other cases, the cohort of interest may include individuals at risk for a specific disease or condition (e.g., pregnancy, HIV, influenza, male androgenetic alopecia, etc.). In some instances, the medical intervention may have been administered or exposed to individuals of the cohort of interest in order to address the specific disease or condition experienced by or affecting the cohort of interest.

As described above, embodiments of the methods include obtaining a plurality of health records including clinical notes regarding individuals associated with the medical intervention. In some instances, individuals associated with the medical intervention are individuals exposed to or receiving the medical intervention. In some instances, individuals associated with the medical intervention are individuals diagnosed with or experiencing a specific disease or condition that may be addressed or treated by the medical intervention. In some instances, individuals associated with the medical intervention are individuals at an elevated risk for a specific disease or condition that may be addressed or treated by the medical intervention. In some instances, individuals associated with the medical intervention are individuals for whom it is possible to develop, or be diagnosed, with a specific disease or condition that may be addressed or treated by the medical intervention.

In some embodiments, the EHR data may be filtered such that only the EHR data necessary to perform the subsequent steps of the invention is transmitted. In some cases, only EHR data (e.g., clinical notes) regarding individuals associated with the medical intervention and belonging to a cohort of interest are transmitted. For example, the transmitting may include applying a computer program or lines of computer code implementing rule-based methods to the structured data of a database that selects the most recent clinical note of individuals exposed to or undergoing a specific medical intervention and passes the notes, and necessary structured EHR data, to a means for performing a subsequent step or steps of the methods (e.g., a processor, computer program or application, lines of computer code, etc.). In some cases, only the history of present illness (HPI) section of each clinical note is transmitted.

As described above, embodiments of the methods include obtaining a plurality of health records including clinical notes regarding individuals associated with the medical intervention. By medical intervention is meant a treatment, procedure, or other action taken to prevent or treat disease, or improve health in any number of a variety of ways. In some embodiments, the medical intervention may include the administration of a pharmaceutical composition, medical device, surgery, and/or therapy to a subject or an alteration of lifestyle (e.g., responsibilities of employment, diet, exercise plans, etc.) by a subject.

In some embodiments, the medical intervention may include the administration of a pharmaceutical composition to a subject. In these instances, the pharmaceutical composition may include any dosage form configured to deliver an active pharmaceutical ingredient (API) of a pharmaceutical composition to a subject. The dosage forms may vary depending on the desired route of administration of a pharmaceutical composition and API therein. By route of administration is meant the way an API enters into an individual's system (e.g., how an API is taken into an individual's body). For example, routes of administration may include, but are not limited to, administrating a pharmaceutical composition and API therein orally, sublingually, topically, transdermally, rectally, vaginally, nasally, optically, by inhalation, and by injection. The pharmaceutical composition may include any number of a variety of APIs that are provided to treat or address (e.g., prevent) any number of a variety of diseases or conditions, such as any of the diseases or conditions described above. These API's include, without limitation, opiates, drugs used in psychiatry or in the treatment of schizophrenia, drugs used for birth control, cytotoxic substances, analgesics, anti-inflammatories, antipyretics, antibiotics, antimicrobials, anxiolytics, laxatives, anorexics, antihistamines, antidepressants, anti-asthmatics, antidiuretics, anti-flatulents, antimigraine agents, antispasmodics, sedatives, steroids, anti-hyperactives, antihypertensives, tranquilizers, decongestants, beta blockers, peptides, proteins, genes and vectors used for gene therapy, oligonucleotides and other substances of biological origin, biologically active organic compounds, and combinations thereof. In some instances, the pharmaceutical composition may include a vitamin, mineral and/or dietary supplement.

In some embodiments, the medical intervention may include the application of a medical device to a subject. In these instances, the medical device may include, but is not limited to, medical devices provided to enhance mobility or communication, regulate biological function, measure or track physiological data, image, correct a biological deficiency or dysmorphic feature, and/or administer a pharmaceutical composition (e.g., as described above). In embodiments where the medical device is provided to enhance mobility or communication, the medical device may be, e.g., a prosthetic (e.g., a prosthetic limb, organ, or a neuroprosthetic such as a pacemaker or cochlear implant), an artificial hip or joint, a physical therapy machine, plates or screws, etc. In embodiments where the medical device is provided to regulate biological function, the medical device may be, e.g., a medical ventilator, incubator, anesthetic machine, heart-lung machine, ECMO, dialysis machine, IUD, etc. In embodiments where the medical device is provided to measure or track physiological data, the medical device may be, e.g., a wearable device (e.g., a smartwatch or a wearable sensor) or an implanted medical device. In embodiments where the medical device is provided to image, the medical device may be, e.g., an ultrasound and/or MRI machine, PET scanner, x-ray machine, etc. In embodiments where the medical device is provided to correct a biological deficiency or dysmorphic feature, the medical device may be, e.g., a medical laser or a surgical machine (e.g., a LASIK surgical machine). In embodiments where the medical device is provided to administer a pharmaceutical composition, the medical device may be, e.g., an insulin pump or a hormonal IUD.

In some embodiments, the medical intervention may include the application of a surgery or therapy to a subject. In these instances, the medical intervention may include, but is not limited to, surgeries and/or therapies provided to treat or address (e.g., prevent) any number of a variety of diseases or conditions, such as any of the diseases or conditions described above. In embodiments where the medical intervention is a surgery, the surgery may affect any organ or organ system of the body. In some instances, the surgery may be, but is not limited to, a cesarean section, organ replacement, joint replacement, hysterectomy, heart surgery, bariatric surgery, organ or tumor removal, brain surgery, etc. In embodiments where the medical intervention is a therapy, the therapy may be, but is not limited to, psychotherapy (e.g., psychodynamic therapy or behavioral therapy), physical therapy, or gene therapy.

In some embodiments, the medical intervention may include a lifestyle change such as, e.g., an exercise plan, a career change, or a dietary change or restriction. In some embodiments, the medical intervention may include a temporary or permanent modification to the subject's responsibilities of employment. In some instances, the medical intervention may include a detoxification process or the wearing of personal protective equipment (PPE).

As described above, the EHR data may regard individuals exposed to or undergoing a medical intervention (such as, e.g., any of the medical interventions described above). In some instances, the EHR data may regard individuals exposed to or undergoing the same medical intervention. In other instances, the EHR data may regard individuals exposed to or undergoing a variety of different medical interventions. In these cases, the individuals may be diagnosed and/or afflicted with the same disease or condition and the medical interventions may be applied to the individuals in order to treat or address the disease or condition. For example, the EHR data may regard individuals diagnosed with inflammatory bowel disease (IBD) and may include a plurality of individuals receiving a first steroid-sparing immunosuppressant to address the IBD, and a plurality of individuals receiving a second steroid-sparing immunosuppressant to address the IBD, and a plurality of individuals receiving a third steroid-sparing immunosuppressant to address the IBD, etc.

In some embodiments, additional electronic data such as, e.g., additional data from digitally available trials or registries may be obtained as discussed above. In some cases, the trial may include a clinical trial. In some embodiments, the structured data of the additional electronic data may be extracted or obtained using, e.g., rules-based approaches implemented using lines of computer code. In some instances, the obtained additional health data may be used to identify an AE of interest, an AE context of interest, or a medical intervention of interest. For example, in embodiments where the additional electronic data includes a clinical trial, AEs (e.g., side effects) identified in the clinical trial may be selected as AEs of interest.

As discussed above, embodiments of the methods include obtaining a plurality of health records including clinical notes regarding individuals associated with a medical intervention. The individuals may include or consist of any number of a variety of cohorts of interest and may be associated with the medical intervention in any number of a variety of way. In some embodiments, the individuals are all diagnosed with the same disease or condition and are receiving or exposed to at least one of a variety of medical interventions to address the disease or condition. The plurality of health records may be obtained through any available means, and from any available source. In some embodiments, the plurality of health records are deidentified EHRs and the EHRs are obtained by transmitting them from a database storing a corpus of EHR data to a means for performing a subsequent step or steps of the methods using lines of computer code. In these instances, the EHR data may be filtered such that only the EHR data necessary to perform the subsequent steps of the invention is transmitted. The obtained EHR data may then be preprocessed such as, e.g., to label medical interventions and adverse events in the plurality of clinical notes as discussed in greater detail below.

### Preprocessing Obtained EHR Data and Clinical Notes

Embodiments of the methods include preprocessing the plurality of clinical notes obtained, e.g., as described above. By preprocessing is meant preparing the plurality of obtained clinical notes for the subsequent steps of the methods as described in greater detail below. In some embodiments, the preprocessing may include labeling medical interventions and adverse events (AEs) in the plurality of clinical notes. In some instances, the preprocessing may include reducing or filtering the plurality of health records.

As discussed above, embodiments of the methods may include reducing or filtering the plurality of health records (e.g., EHR data). By reducing or filtering is meant decreasing the amount of data without hindering the accuracy or efficiency of the methods (i.e., without negatively effecting the trained machine learning model). In some embodiments, the obtained EHR data may be reduced or filtered such that data determined to not be necessary to perform the subsequent steps of the invention is omitted or excluded. In these instances, the data omitted or excluded may vary, and may depend on, e.g., tasks the machine learning model is trained to perform, AEs of interest, AE contexts of interest, medical interventions of interest, cohorts of interest, a time window determined to be compatible with a causal relationship between an AE and a medical intervention, and a desired accuracy or efficiency of the machine learning model. For example, in embodiments where medical interventions for the treatment of a disease or condition are compared, obtained EHR data may be reduced to only include the EHR data (e.g., clinical notes) of individuals diagnosed with the disease or condition and/or receiving a medical intervention to address the disease or condition. In some embodiments, the clinical notes obtained for each subject may be filtered or reduced to avoid redundancies and/or increase efficiency. In these instances, the clinical notes obtained for each individual may be filtered or reduced to only include the most recent clinical note for each individual or, if the individual has ceased receiving the medical intervention, the most recent clinical note before a time window determined to be compatible with a causal relationship between an AE and a medical intervention has passed following the conclusion of the medical intervention. In some cases, the clinical notes obtained for each subject may be filtered or reduced to only include a specific section of a clinical note such as, e.g., the history of present illness (HPI) section of each clinical note. In embodiments where obtaining the EHR data includes transmitting the data, the reducing or filtering may occur before or after transmission occurs (e.g., as discussed above).

As discussed above, embodiments of the methods include labeling or tagging components in the plurality of clinical notes. By labeling or prelabeling is meant identifying a component (e.g., a medical intervention or AE) within the plurality of clinical notes and adding or attaching one or more meaningful and informative labels or tags providing context that enables the machine learning model to learn from the component. In some embodiments, the preprocessing includes the labeling of medical interventions and AEs in the plurality of clinical notes. By adverse event (AE) is meant a harmful or negative occurrence in an individual receiving, or exposed to, a medical intervention, that may or may not be associated with (e.g., caused by) the medical intervention. Adverse events can include any unfavorable and/or unintended sign, symptom, disease and/or condition occurring in (i.e., experienced by) an individual exposed to the medical intervention. In some embodiments, the AEs may include, but are not limited to, any of the over 80,000 AEs used in AE reporting in the context of medical product regulation disclosed by the Medical Dictionary for Regulatory Activities (MedDRA). In some embodiments, the AEs may include, but are not limited to, any of the AEs or adverse reactions or effects found in the Medical Subject Headings (MeSH) thesaurus, the International Classification of Diseases, Tenth Revision, Clinical Modification (ICD-10-CM) system or the WHO Adverse Reactions Terminology (WHO-ART) dictionary.

The AEs that are labelled may vary and may be found, or may affect, any organ or organ system of the body. In some embodiments, the AEs may include one or more infections and infestations. For example, the AEs may include a urinary tract infection, an abscess, a staphylococcal infection and/or appendicitis. In some embodiments, the AEs may include any of the signs, symptoms, or diseases and/or conditions associated with lack of intended medical intervention efficacy. AEs associated with lack of intended medical intervention efficacy may depend on, e.g., the disease or condition for which the medical intervention is provided to address. For example, in individuals diagnosed with IBD wherein the medical intervention is receiving a steroid-sparing immunosuppressant to address the IBD, AEs associated with lack of intended medical intervention efficacy may include, e.g., inflammation, proctitis, ulcerative colitis, abdominal pain, diarrhoea, constipation, and/or peritonitis.

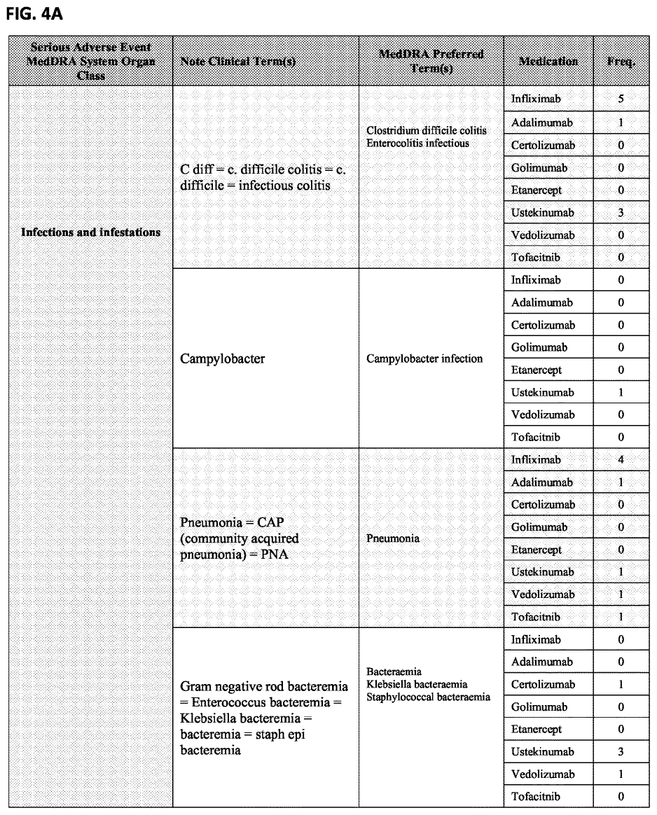

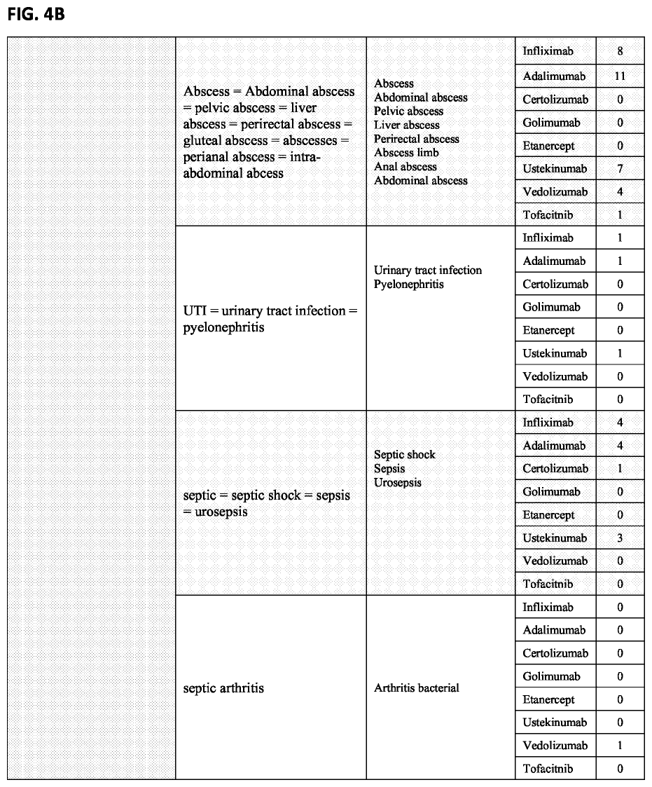

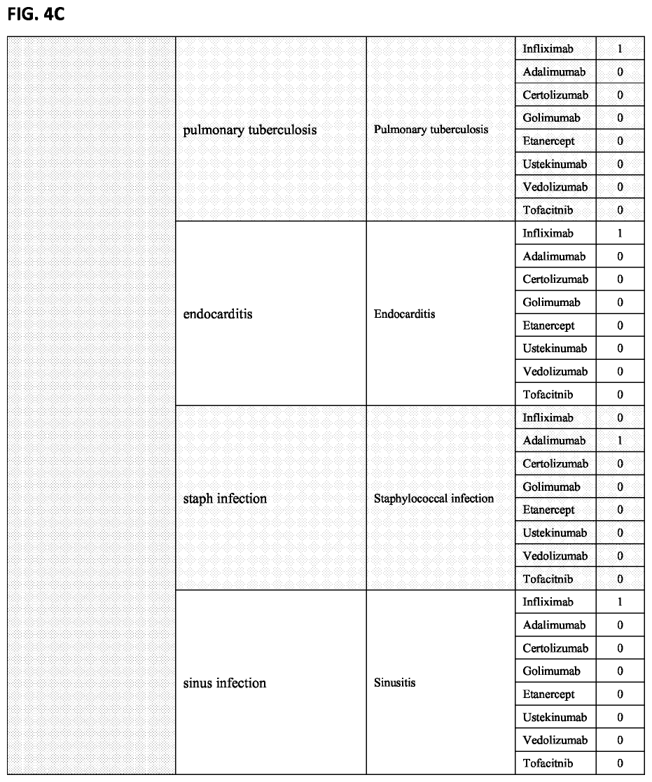

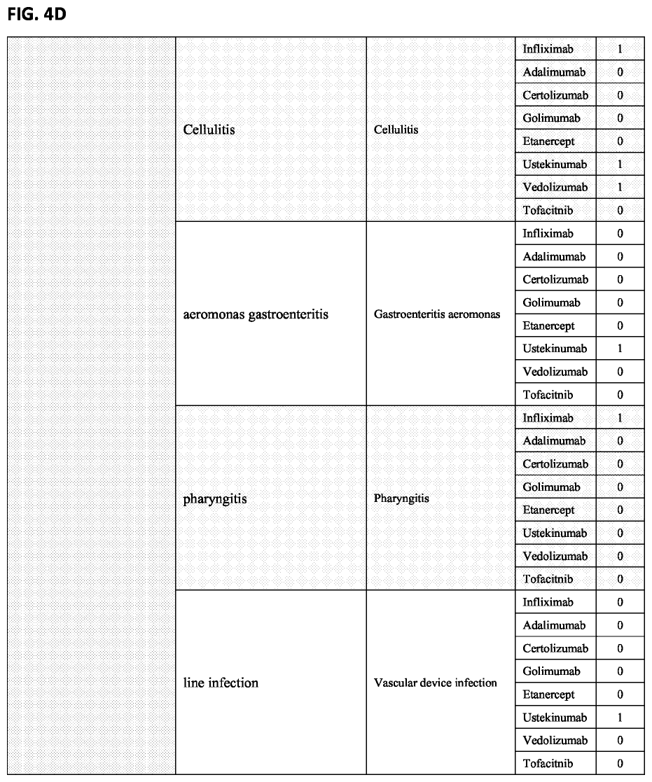

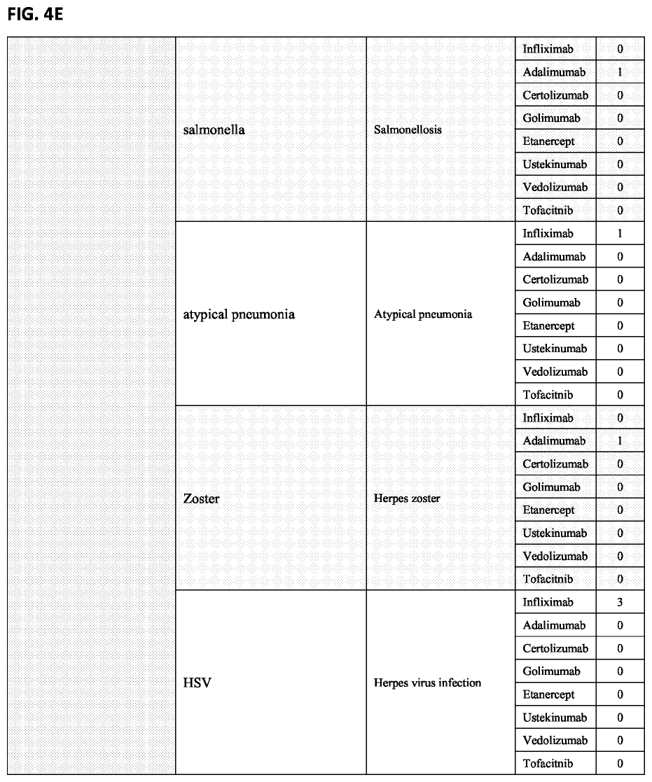

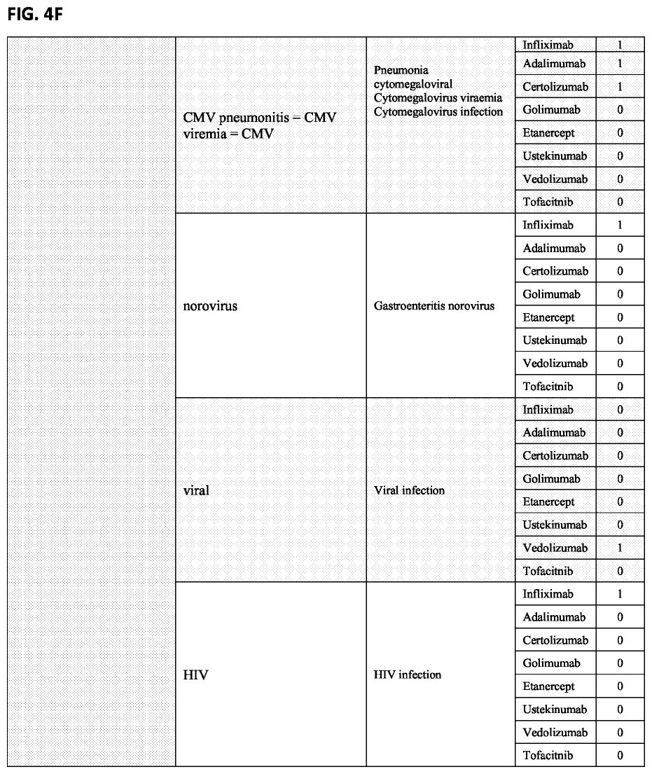

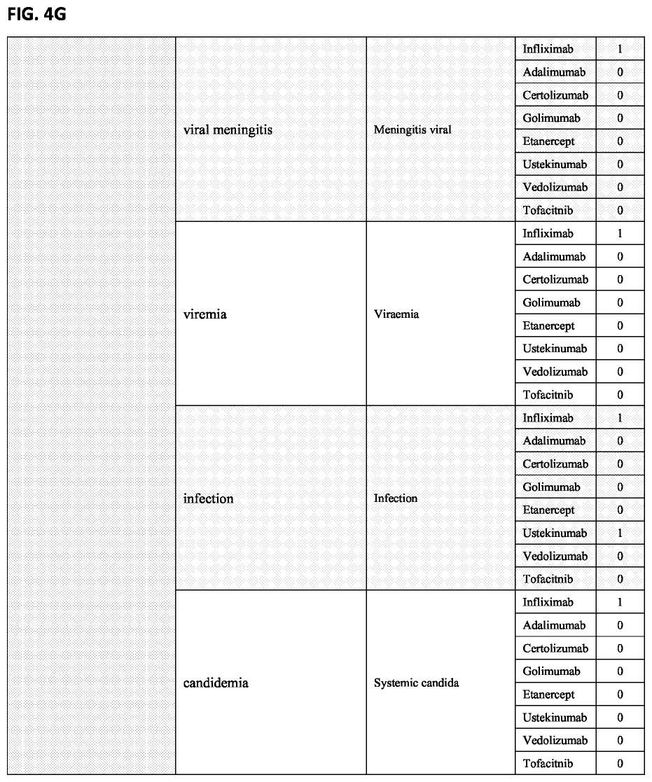

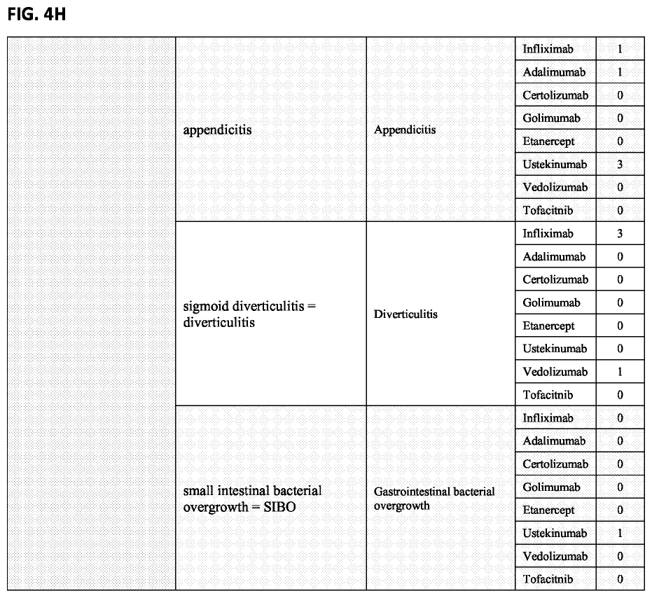

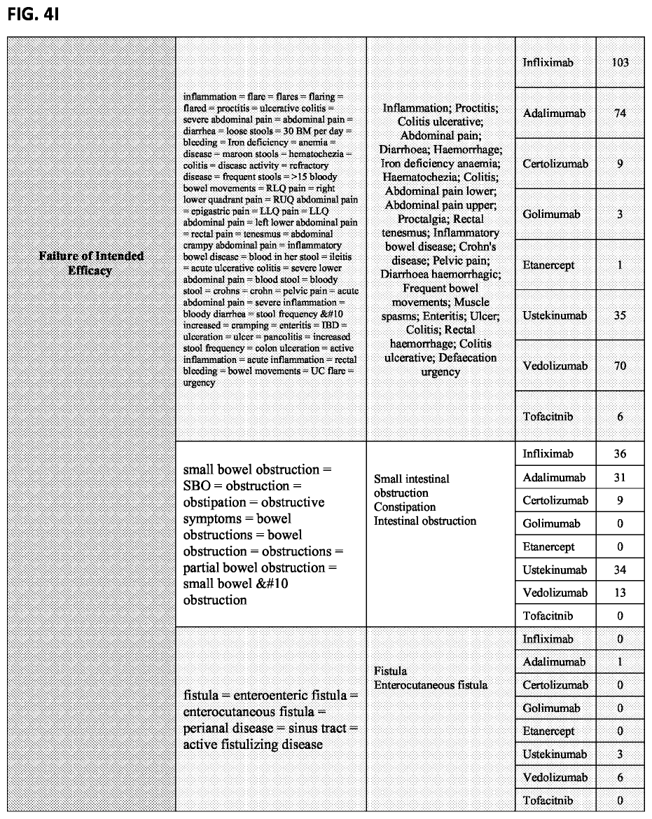

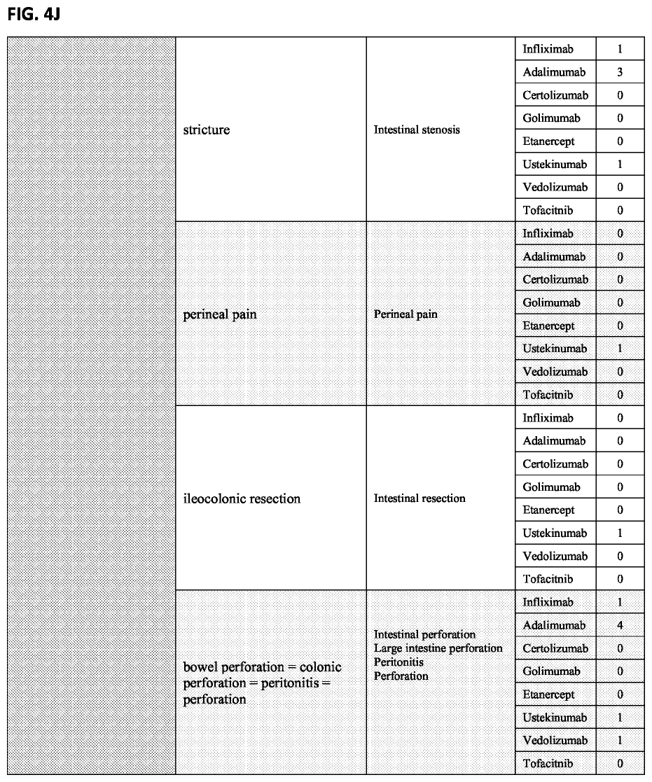

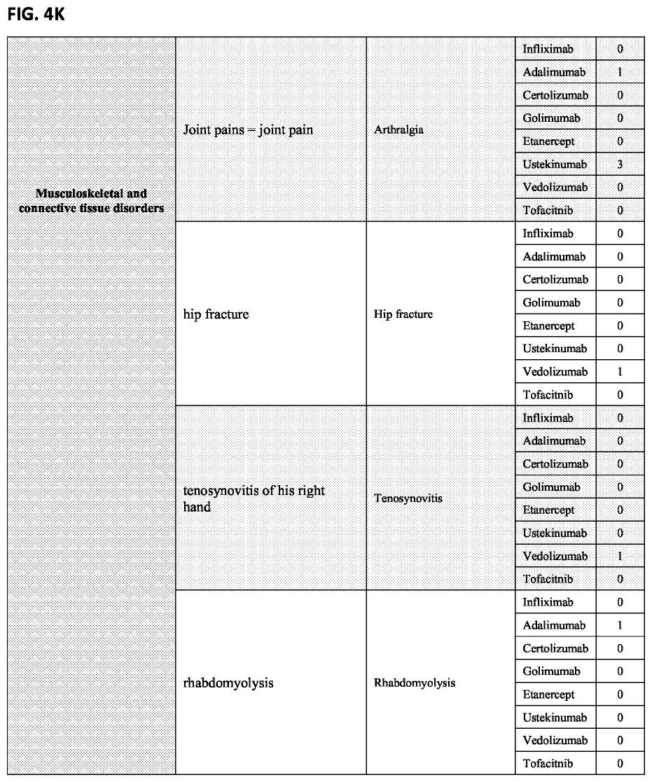

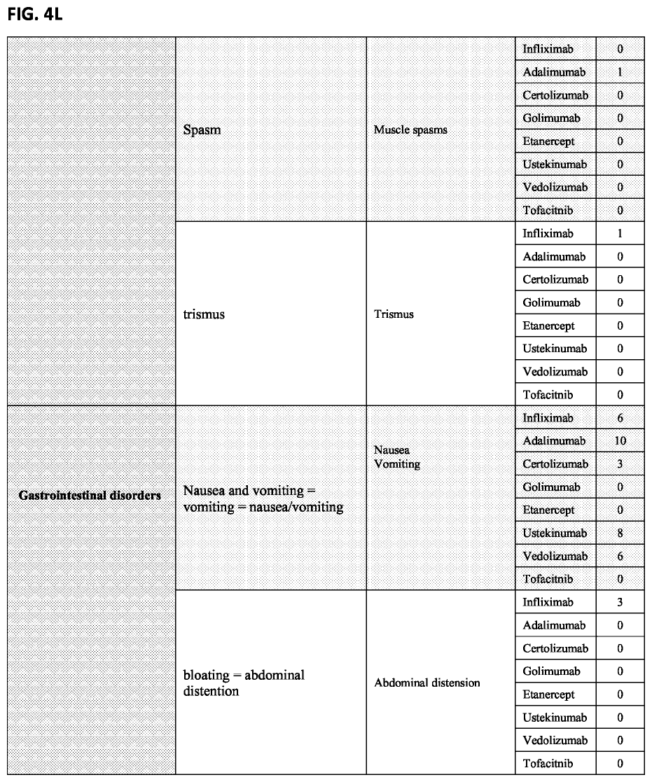

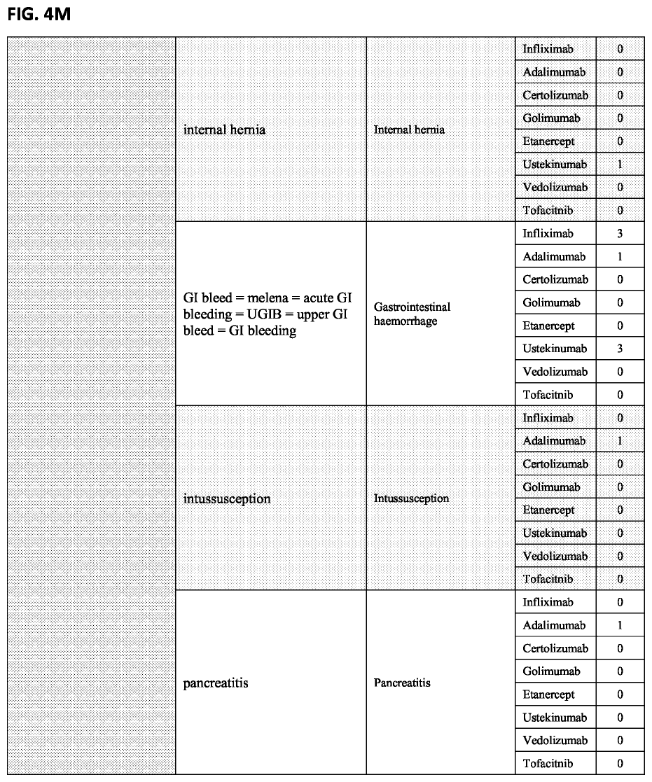

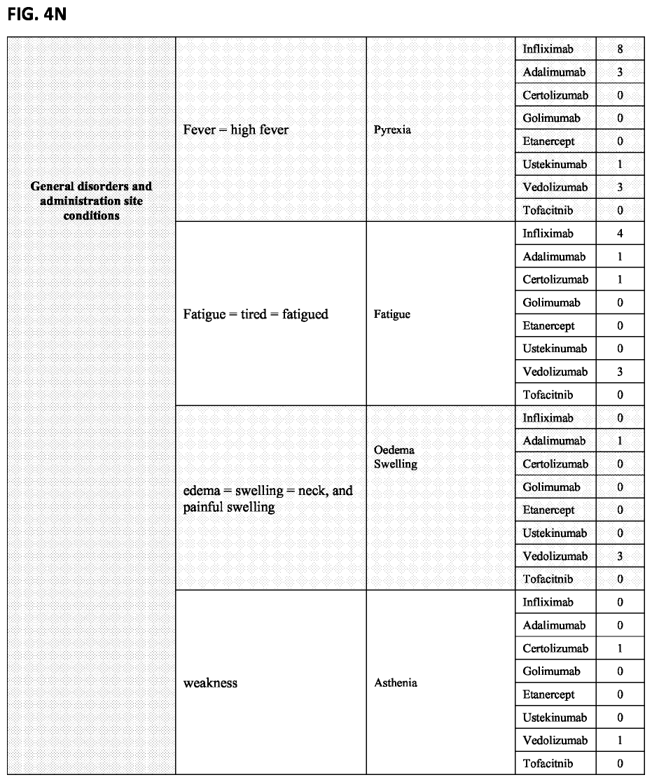

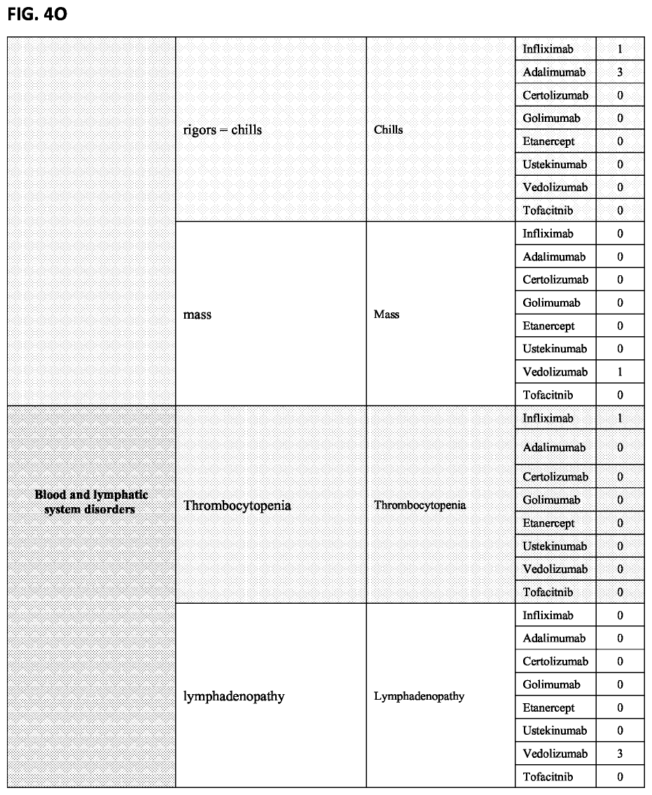

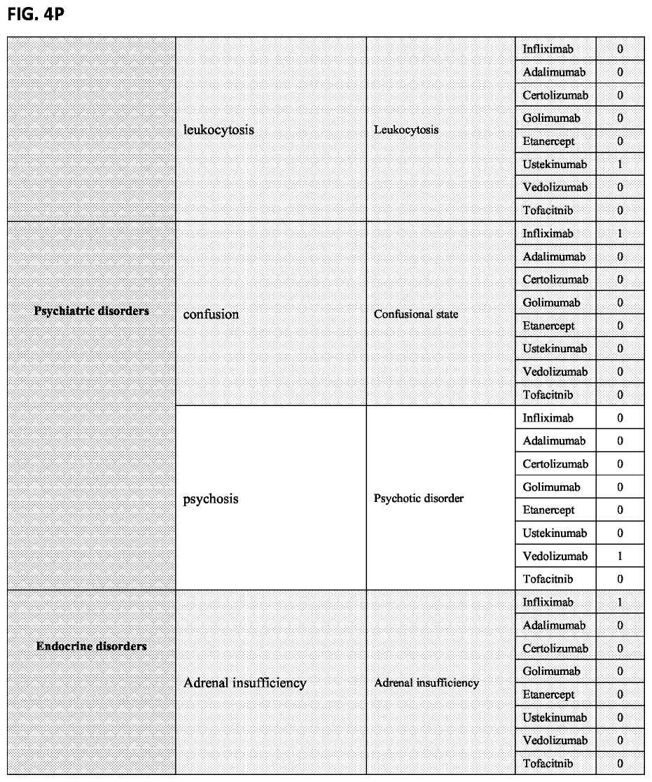

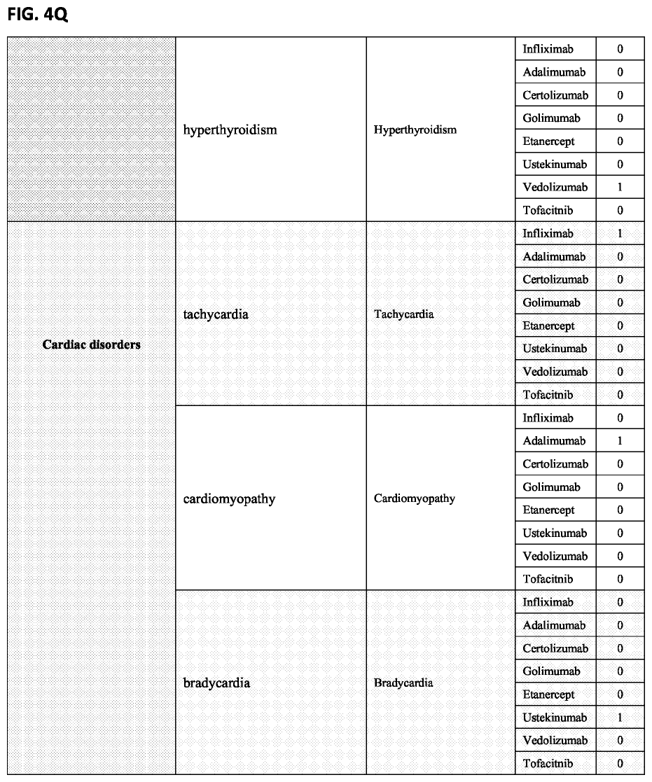

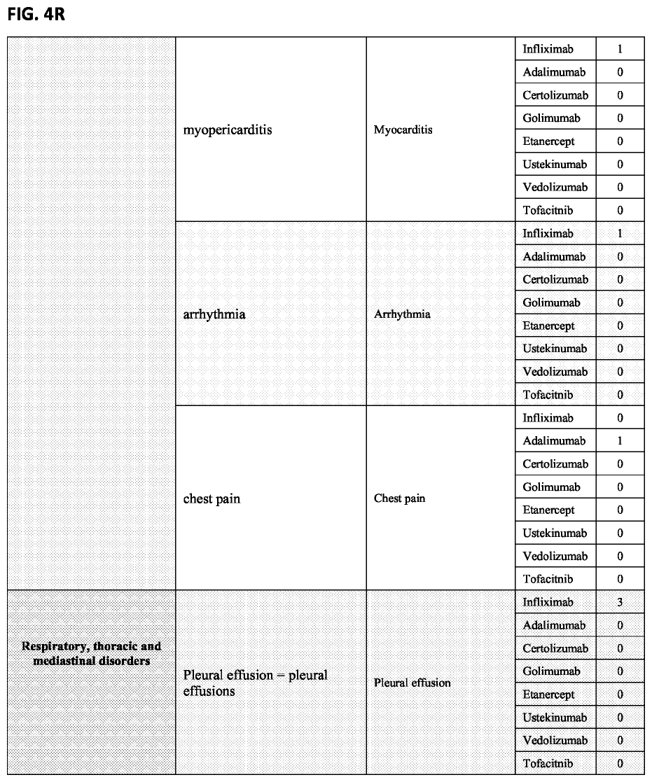

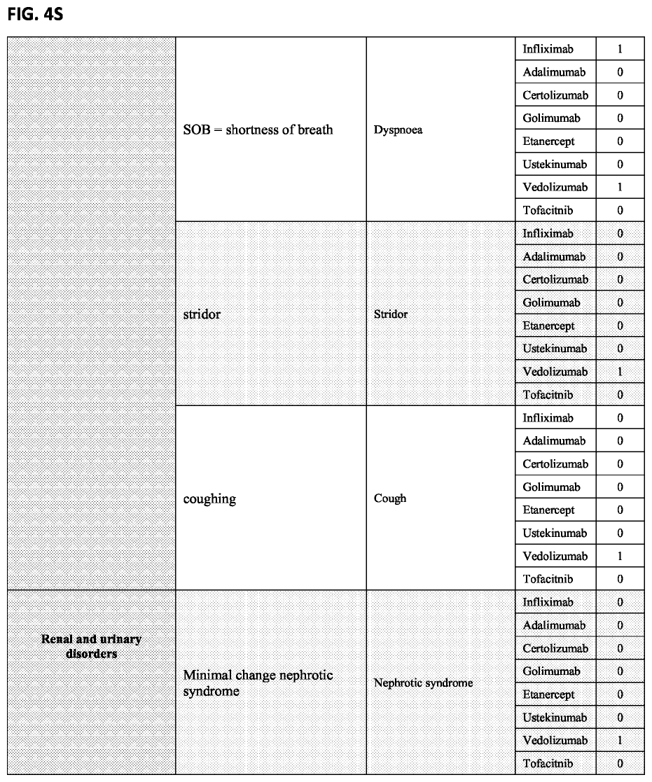

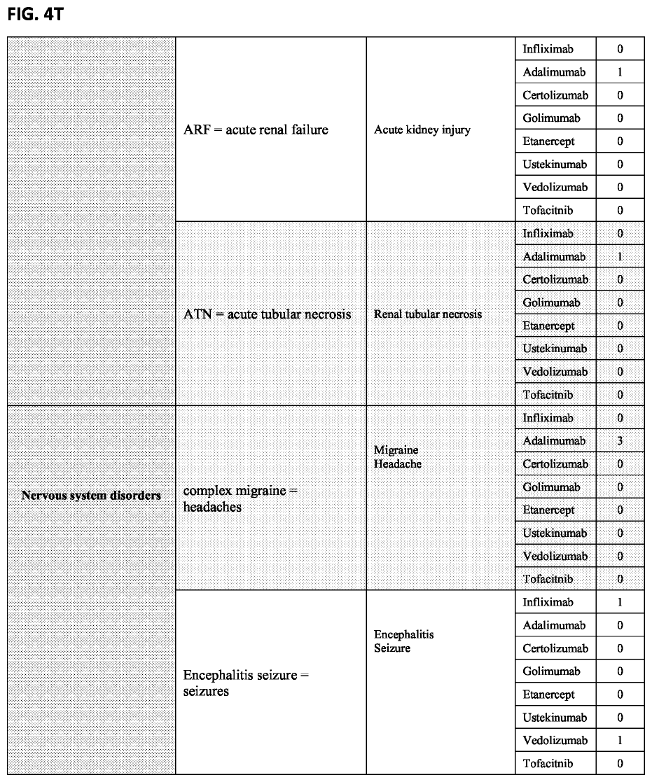

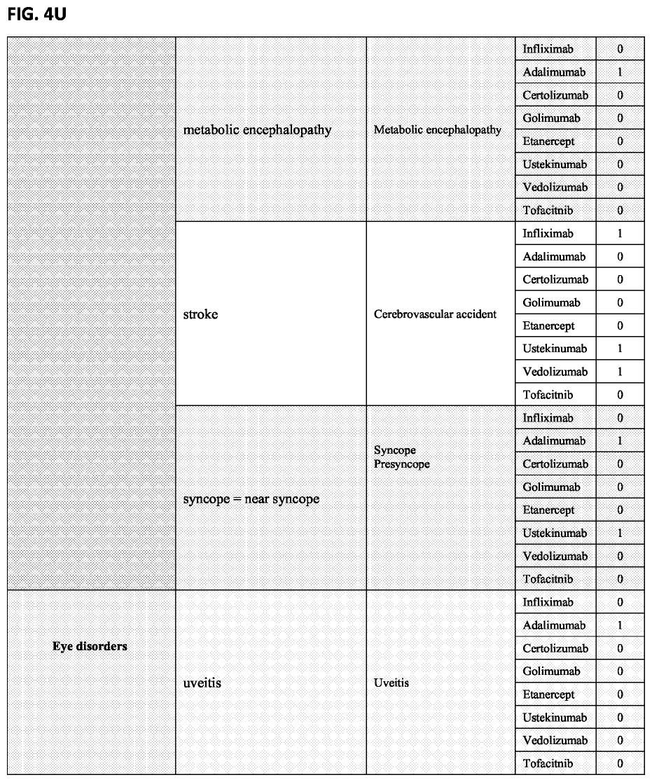

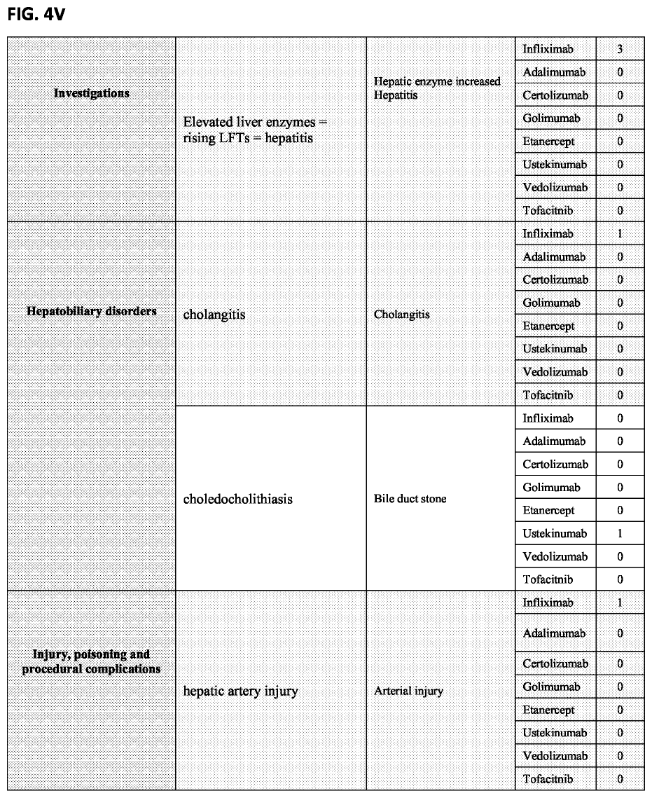

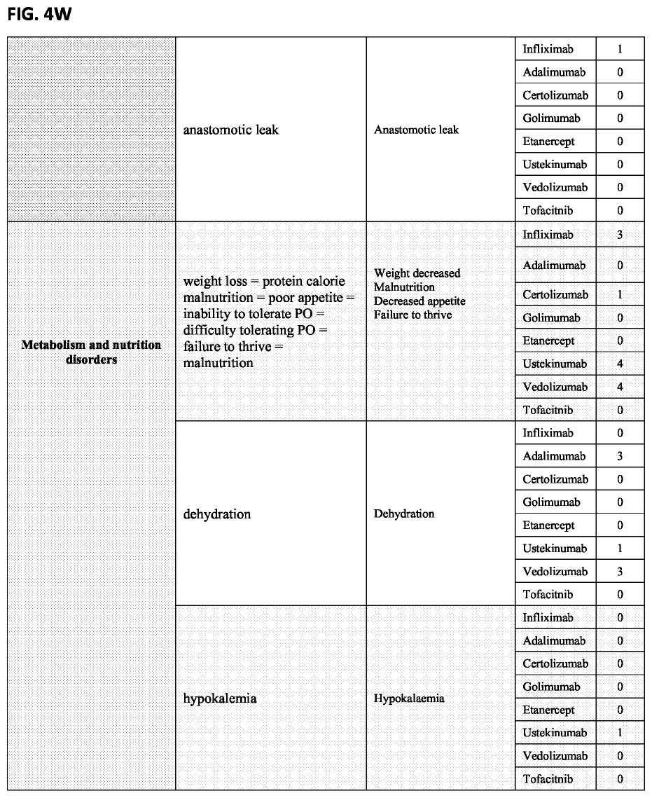

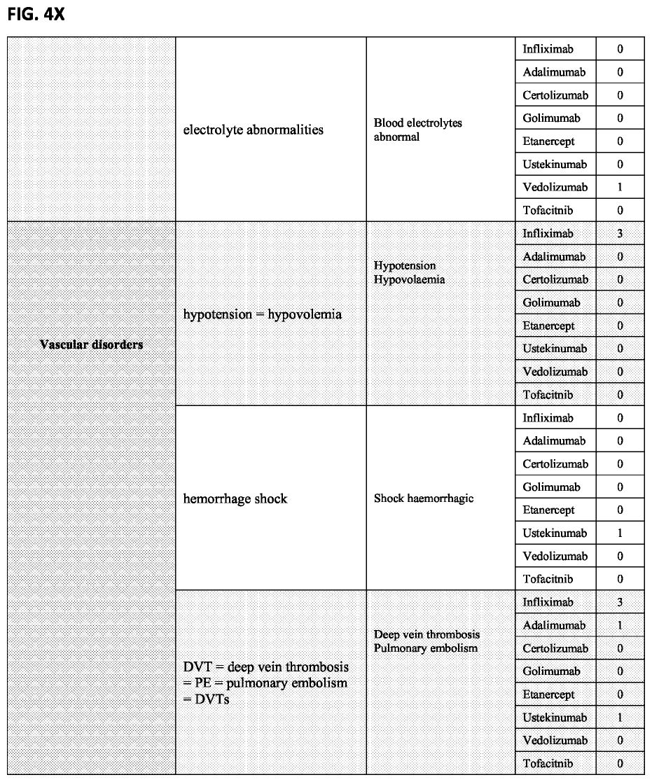

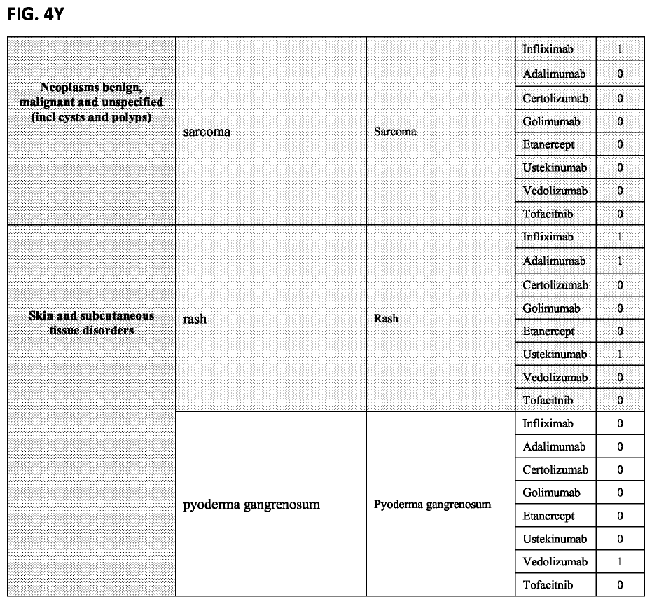

In some embodiments, the AEs may include one or more musculoskeletal and/or connective tissue disorders. For example, the AEs may include arthralgia, a hip fracture, and/or tenosynovitis. In some embodiments, the AEs may include one or more gastrointestinal disorders. For example, the AEs may include nausea, an internal hernia, and/or pancreatitis. In some embodiments, the AEs may include one or more general disorders and/or administration site conditions. For example, the AEs may include fatigue, swelling, and/or chills. In some embodiments, the AEs may include one or more blood and lymphatic system disorders. For example, the AEs may include thrombocytopenia and/or leukocytosis. In some embodiments, the AEs may include one or more psychiatric disorders, endocrine disorders, cardiac disorders, respiratory, thoracic and/or mediastinal disorders, renal and/or urinary disorders, nervous system disorders, eye disorders, inflammation, hepatobiliary disorders, injuries, poisoning and/or procedural complications, metabolism and/or nutrition disorders, vascular disorders, neoplasms and/or skin and subcutaneous tissue disorders, etc. In some embodiments, the AEs may include, but are not limited, any of the AEs found in FIG. 4A to 4Y.

In some embodiments, the preprocessing may further include the labeling or identification of one or more contexts of interest. By contexts of interest is meant one or more circumstances or factors that, when associated with an AE, are indicative of the nature or severity of the AE. In some cases, a context of interest may be a location in which the individual is receiving treatment such as, e.g., a hospital or the specific department of a hospital, a doctor's office, an infusion center, a dialysis center, etc. In some instances, a context of interest may be a patient outcome such as, e.g., death, the requiring of an additional medical intervention (i.e., other than the medical intervention of interest for which AEs are being identified), permanent damage or disfigurement, etc. In some instances, a context of interest may be the nature of or reason for an interaction of a healthcare professional with an individual such as, e.g., birth encounters, routine checkups, medical testing appointments, emergency encounters, etc. In some embodiments, contexts of interest are identified and labeled in the clinical notes or identified in and associated with a clinical note (e.g., used to classify or label a clinical note as a whole). In some embodiments, contexts of interest are identified and/or labeled in the structured EHR data associated with a clinical note using, e.g., lines of computer code and rule-based methods. In these instances, an identified or labeled context of interest may be used to classify or label a clinical note as a whole. In some embodiments, identified or labeled contexts of interest may be used to filter or reduce the EHR data to only include clinical notes (and, e.g., the structured EHR data associated therewith) where the context or contexts of interest have been identified. In these cases, the filtering may occur before or after transmission occurs (e.g., as discussed above).

In some embodiments, the AE may be a serious adverse effect. By serious adverse effect (SAE) is meant an AE that results in death, is life-threatening, requires inpatient hospitalization or causes prolongation of existing hospitalization, results in persistent or significant disability/incapacity, may have caused a congenital anomaly/birth defect, and/or requires intervention to prevent permanent impairment or damage. In some cases, contexts of interests may be one or more of the contexts that would classify an AE as an SAE if associated therewith (e.g., hospitalization, a congenital anomaly/birth defect, significant disability/incapacity, intervention to prevent permanent impairment or damage, death, etc.). In some embodiments, the subsequent steps of the methods (e.g., the annotating and the training and applying of the machine learning model) as described in greater detail below may determine if the AE is associated with one or more contexts of interest and/or if the AE is an SAE. For example, the preprocessing may include the labeling of medical interventions, AEs, and hospitalizations. In these instances, the subsequent steps of the methods may determine if the AE is associated with the hospitalization (e.g., if the AE may have, or did, cause or result in the hospitalization).

In some embodiments, all AEs are labeled or tagged. In other instances, only AEs of interest are labeled or tagged. In some cases, specific AEs, such as those identified in other studies or in clinical trials, may be labeled or tagged with additional information or context, e.g., indicative of the nature or severity of the AE or that the AE was previously identified as being associated with (e.g., caused by) a medical intervention of interest. In some cases, AEs not previously identified as being associated with (e.g., caused by) a medical intervention of interest may be labeled or tagged with information or context indicating that they have not previously been associated with a medical intervention of interest in prior studies or trials. In some embodiments, all medical interventions are labeled or tagged. In other instances, only medical interventions of interest are labeled or tagged. In these cases, the medical interventions may have been applied to the individuals (i.e., for which the plurality of health records is obtained) in order to treat or address a specific disease or condition experienced by or affecting the individuals.

As discussed above, embodiments of the methods include labeling or tagging, among other things, medical interventions, AEs, and contexts of interest in the plurality of clinical notes. In some embodiments, the labelling or tagging may occur automatically using, e.g., lines of computer code or a computer program or system (e.g., one or more functions from a computer program/system). In these instances, the labelling or tagging may occur automatically using one or more functions of a natural language processing software such as, e.g., a clinical text natural language processing software. For example, one or more functions of the clinical Text Analysis and Knowledge Extraction System (cTAKES) software, the Health Information Text Extraction (HITEx) software, and/or the MetaMap program may be used to automatically perform the labelling or tagging of the methods. In some embodiments, one or more functions from the cTAKES software are utilized in order to automatically perform the labelling or tagging. In some instances, the clinical notes may be in a language other than English. In these cases, the natural language processing software utilized to automatically perform the labelling or tagging may vary depending on the language of the clinical notes. For example, when the clinical notes are in, e.g., the French language, one or more functions of the SIFR and/or ECMT softwares may be used to automatically perform the labelling or tagging of the methods. In some cases, a metathesaurus, such as the Unified Medical Language System (UMLS) metathesaurus, may be used, at least in part, to automatically perform the labelling or tagging of the methods. In some instances, the labeling may be performed using rule-based approaches or using supervised machine learning.

In some embodiments, the preprocessing may further include generalizing or normalizing one or more of the labeled (e.g., as described above) components of the plurality of clinical notes. By normalize is meant standardizing equivalent/synonymous or closely related terms or expressions (i.e., components) to a single term or expression. In some cases, labeled medical interventions may be normalized. For example, in embodiments where the medical intervention includes one or more medications, the medications may be normalized to their generic names. In some embodiments, one or more of the labeled components may be normalized to a controlled vocabulary such as, e.g., the controlled vocabulary of a particular database or system. The controlled vocabulary database or system may vary depending on the labeled component being normalized. For example, in embodiments where the medical intervention includes one or more medications or pharmaceutical compositions, the labeled medications or pharmaceutical compositions may be normalized using the controlled vocabulary of the RxNorm system provided by the National Institutes of Health (NIH). In embodiments where the medical intervention includes one or more foods, the labeled foods may be normalized using the controlled vocabulary of the FoodOn ontology. The normalizing may be performed automatically using lines of computer code. In these instances, the normalizing may be performed using rule-based approaches (e.g., cTAKES) or using supervised machine learning.

As discussed above, embodiments of the methods include preprocessing the plurality of obtained clinical notes (i.e., EHR data). The preprocessing may include reducing or filtering the EHR data such that data determined to not be necessary to perform the subsequent steps of the invention, as described in greater detail below, is omitted or excluded. In some embodiments, the EHR data is filtered or reduced to only include the most relevant clinical note from individuals belonging to a cohort of interest. In embodiments of the methods, the preprocessing includes labeling or tagging components in the plurality of clinical notes. In some embodiments, AEs, medical interventions, and contexts of interest are labeled in each clinical note. For example, all AEs, medical interventions of interest, and hospitalizations may be tagged in each clinical note. The labelling or tagging may be performed automatically using lines of computer code and a natural language processing software such as cTAKES. In these instances, rule-based approaches such as, e.g., an NLP pipeline may be used for the labelling or tagging. In some embodiments, the preprocessing may further include generalizing or normalizing one or more of the labelled components of the plurality of clinical notes. Normalizing may occur automatically using lines of computer code and a controlled vocabulary database or system such as RxNorm. In these instances, rule-based approaches (e.g., cTAKES) and/or supervised machine learning may be used for normalizing. The preprocessed clinical notes may then annotated in order to train a machine learning model to perform one or more tasks, as discussed in greater detail below.

### Annotating the Preprocessed Clinical Notes

Embodiments of the methods include annotating a first subset of the plurality of preprocessed clinical notes (e.g., labelled as described above) in order to train a machine learning model to perform one or more tasks, as discussed in greater detail below. By annotating is meant marking (e.g., labeling, tagging, transcribing, or processing) each clinical note with the features the machine learning model is trained to identify, the relationship between prelabeled components (e.g., AEs and medical interventions) the machine learning model is trained to infer, or the outcome of a predictive task the machine learning model is trained to predict. In some embodiments, a first subset of the plurality of preprocessed clinical notes is annotated to indicate if each labeled adverse effect is associated with the medical intervention.

The preprocessed clinical notes may be annotated in a manner sufficient to train a machine learning model to perform any task associated with pharmacovigilance, such as any task improving the availability and quality of information related to medical intervention (e.g., drug or medical device) safety. In some cases, the task may be any aiding or assisting in the monitoring of medical intervention safety or efficacy such as any task aiding in the characterization or validation of a medical intervention safety profile and/or the identification of previously unrecognized AEs or SAEs associated with a medical intervention. In some embodiments, the preprocessed clinical notes may be annotated in a manner sufficient to train a machine learning model to perform one or more tasks aiding in the identification of a medical intervention related AE. In these instances, the identification of the medical intervention related AE may result from the outcomes or outputs of a plurality of tasks performed by a trained machine learning model. In other words, in order to train a machine learning model to perform a task, the task may be broken down or divided into a plurality of subtasks. The subtasks may be annotated (e.g., as separate relationships), trained for, and/or performed separately. The outcomes or outputs from each subtask may then be used to generate an outcome or output for the overall task. In order to aide or assist in the monitoring of medical intervention safety or efficacy, such as through assisting in the identification of medical intervention related AEs, the annotating may include, but is not limited to, annotating the preprocessed clinical notes in a manner sufficient to train a machine learning model to perform any of the tasks described below.

In some embodiments, clinical notes may be annotated to indicate a relationship between a medical intervention and a context of interest. In these instances, the relationship between the medical intervention and the context of interest may vary and may include, but is not limited to, a temporal relationship or a causal relationship. For example, clinical notes may be annotated to indicate if hospitalization (or, e.g., another context of interest) occurred before or after an individual was exposed to or administered a medical intervention and/or before or after the individual ceased receiving, or being exposed to, the medical intervention. In these cases, the individual may be considered to have ceased receiving, or being exposed to, the medical intervention if a time window determined to be compatible with a causal relationship between an AE and a medical intervention has passed after the individual has stopped receiving, or being exposed to, the medical intervention. In some cases, medical interventions of interest may be linked or associated with contexts of interest during annotation if the medical intervention and the context reflect a specific temporal or causal relationship. For example, all hospitalizations occurring after an individual was exposed to or administered a medical intervention of interest and before the individual stopped receiving the medical intervention of interest may be linked or associated with the medical intervention of interest and/or all hospitalizations determined to be caused by a medical intervention of interest may be linked to the medical intervention of interest.

In some embodiments, clinical notes may be annotated to indicate a relationship between an AE and a context of interest. In these instances, the relationship between the AE and the context of interest may vary and may include, but is not limited to, a temporal relationship or a causal relationship. For example, clinical notes may be annotated to indicate if an AE occurred during or before a specific hospitalization and/or if the AE was the cause of the hospitalization. In some cases, AEs of interest may be linked or associated with contexts of interest during annotation if the AE and the context reflect a specific temporal or causal relationship. For example, all hospitalizations occurring as the result of an AE of interest may be linked or associated with the AE of interest.

In some embodiments, clinical notes may be annotated to indicate a relationship between an AE and a medical intervention of interest. In these instances, the relationship between the AE and the context of interest may vary and may include, but is not limited to, a causal relationship. In some embodiments, clinical notes may be annotated to indicate a relationship between an AE of interest, a medical intervention of interest, and a context of interest. For example, clinical notes may be annotated to indicate if a medical intervention of interest occurred before a hospitalization that was caused by an AE of interest.

As discussed above, embodiments of annotating a first subset of the plurality of clinical notes may vary, and may depend on, e.g., the tasks the machine learning model is trained to perform, the AEs of interest, the AE contexts of interest, the medical interventions of interest, the cohorts of interest, the time window determined to be compatible with a causal relationship between an AE and a medical intervention, and the desired accuracy or efficiency of the machine learning model. In some embodiments, clinical notes may be annotated to indicate a relationship between a disease or condition of interest (e.g., a disease or condition diagnosed in a cohort of interest) and an AE of interest. In instances where clinical notes are annotated to indicate a causal relationship, all causes may be annotated. For example, in embodiments where clinical notes are annotated to indicate if a hospitalization was caused by an AE of interest, the clinical notes may be annotated to indicate all causes or reasons for a hospitalization. In instances where an annotator cannot determine the nature of a relationship with certainty, clinical notes may be annotated to indicate a possible or likely relationship. For example, in embodiments where clinical notes are annotated to indicate a relationship between a medical intervention and an AE of interest, the clinical notes may be annotated to indicate a possible or likely causal relationship between the medical intervention and the AE (i.e., to indicate it is possible the medical intervention caused or resulted in the AE).

The annotating may be performed by one or more individuals having the knowledge and ability to annotate the clinical notes with sufficient accuracy. By sufficient accuracy in this context is meant the created annotations, when taken together, are capable of training a machine learning model to perform a desired task with a desired accuracy, speed, and/or efficiency. In some instances, the annotating may be performed by one annotator. In other cases, the annotating may be performed by more than one annotator, such as two or more annotators, or three or more, or five or more, or ten or more, or twenty or more. In some embodiments, the one or more annotators may have specialized knowledge, training, education or experience relevant to the determinations made when annotating. For example, the annotators may have education or experience regarding AEs of interest, AE contexts of interest, medical interventions of interest, cohorts of interest, etc. In some embodiments, the annotators may be medical professionals, pharmacovigilance specialists, and/or patients. In some embodiments, the annotating may be performed, at least in part, automatically using lines of computer code. In some cases, the annotating may be crowd sourced. In these instances, the crowd sourcing may include random individuals or may be limited to a cohort of interest (e.g., doctors, medical students, patients, etc.)

The annotations (i.e., indicative of any of the relationships, features, or outcomes described above) may be generated or created through any number of various methods, systems, or protocols. In some embodiments, the one or more annotators may develop an annotation protocol in order to efficiently and accurately annotate a first subset of the plurality of preprocessed clinical notes. The annotation protocol may vary depending on, e.g., the tasks the machine learning model is trained to perform, the AEs of interest, the AE contexts of interest, the medical interventions of interest, the cohorts of interest, the time window determined to be compatible with a causal relationship between an AE and a medical intervention, and the desired accuracy or efficiency of the machine learning model. In some cases, the annotation protocol may be collectively developed by multiple annotators and may be refined one or more times. In some embodiments, a measurement or assessment may be performed to assess the quality, accuracy, or agreement between annotations. In these instances, the assessment may be an interrater reliability assessment including, but not limited to, Cohen's kappa, Scott's pi, Fleiss' kappa, inter-rater correlation, concordance correlation coefficient, intra-class correlation, and/or Krippendorff's alpha statistics. In some embodiments, the interrater reliability assessment may be a Fleiss' kappa statistic. In some embodiments, the interrater reliability assessment (e.g., the Fleiss' kappa statistic) is used to determine if the annotation protocol is refined, if more annotations are generated, and/or if new annotators are selected.

The annotations may be generated or created through any number of various means. In some embodiments, an annotation platform (such as, e.g., a computer software or application) may be used that allows an annotator to efficiently perform annotating (i.e., generate or create annotations). For example, annotating may be performed using the Diffgram, Label Studio, Labelbox, or Datasaur programs/platforms, or similar programs and platforms thereof. In some embodiments, the annotation platform is Label Studio.

As discussed above, embodiments of the methods include annotating a first subset of the plurality of preprocessed clinical notes (e.g., labelled as described above) in order to train a machine learning model to perform one or more tasks. The preprocessed clinical notes may be annotated in a manner sufficient to train a machine learning model to perform any task associated with pharmacovigilance, including tasks aiding in the identification of a medical intervention related AE. In some embodiments, clinical notes may be annotated to indicate a relationship between medical interventions of interest, contexts of interest (e.g., hospitalization, congenital birth defects, or death), and/or AEs of interest. The annotating may be performed by one or more individuals having the knowledge and ability to annotate the clinical notes with sufficient accuracy. For example, the annotators may include medical professionals, pharmacovigilance specialists, and/or patients. The annotations may be generated or created through any number of various methods or protocols. In some embodiments, the annotators may develop an annotation protocol that results in annotations that meet a predetermined threshold of interrater reliability as measured through a Fleiss' kappa statistic. The annotations may be generated or created using an intuitive and efficient annotation platform such as LabelStudio. The annotated clinical notes may then be used to train a machine learning model to train a machine learning model to perform one or more tasks, as discussed in greater detail below

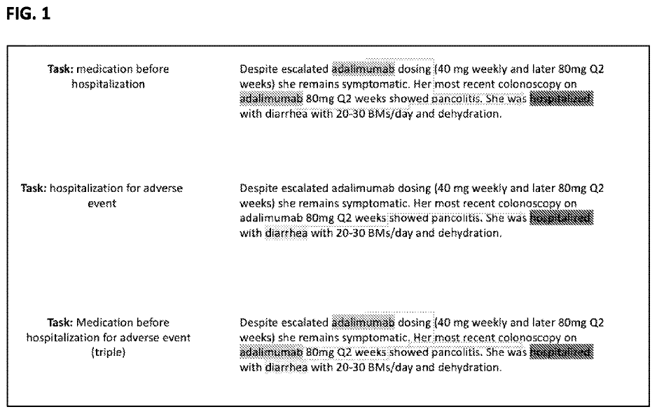

FIG. 1 provides an excerpt of a clinical note annotated in three different manners in order to train a machine learning model to perform three different predictive tasks. Medications of interest (i.e., medical interventions of interest) are labeled in blue, hospitalizations (i.e., contexts of interest) in red and signs and symptoms (i.e., AEs of interest) in yellow. The annotations depicted train a machine learning model to perform prediction tasks and subtasks enabling the machine learning model to be used for: classifying whole HPIs according to the occurrence of at least one documented SAE, classifying candidate medication-hospitalization or hospitalization-AE pairs as to whether or not they belong to a valid SAE triple relationship, and classifying candidate medication-hospitalization-AE triples as to whether or not they are a valid SAE.

### Training the Machine Learning Model

Embodiments of the methods include training a machine learning model to identify adverse events associated with a medical intervention using the annotated clinical notes (e.g., annotated as described above). By training is meant providing or feeding the annotated clinical notes to the machine learning model so that the model can adjust one or more of its components (e.g., weights or biases) in order to or effectively (e.g., accurately or efficiently) perform a task. The machine learning model, in accordance with embodiments of the methods, may vary and may include, but is not limited to, any of the models discussed below. In some embodiments, the training may further include pretraining, validating, and testing.

In some embodiments, the machine learning model may include an artificial neural network (NN) (e.g., a convolutional NN (CNN)). In some embodiments, the machine learning model is a deep learning model. In these cases, the model may be three or more layers deep, such as five or more layers deep, or ten or more, or twelve or more, or sixteen or more, or twenty-four or more, or thirty-two or more, or sixty-four or more. In some embodiments, the machine learning model is configured to process sequential input data. In these instances, the machine learning model may include, or be based on, a recurrent neural network (RNN) model or a transformer model. In embodiments where the machine learning model includes an RNN, the RNN may include, e.g., long short-term memory (LSTM) architecture and/or gated recurrent units (GRUs). In some embodiments, the machine learning model may include, or be based on, the architecture of a transformer model.

As discussed above, the machine learning model may be configured to process sequential input data. The sequential input data may be a sequence of words, and the machine learning model may be configured to use the bag-of-words (BoW) model and/or word embeddings to represent the sequence of words. In embodiments using the BoW model, the machine learning model may include a Logistic Regression, K-Nearest Neighbors, Decision Tree, Random Forest and/or XGBoost model. In some embodiments, the machine learning model may use word embeddings to represent the sequence of words. In these instances, the machine learning model may be configured to learn from the contextual information of a word (i.e., the words before or after a given word sequentially). The machine learning model may learn from the contextual information of a word using contextual word embeddings and, e.g., may learn from the left to right context of a word and/or the right to left context of a word. In some embodiments, the machine learning model may learn from both the left to right context and the right to left context of a word (i.e., the machine learning model may be bidirectional). For example, the machine learning model may include, or be based on, a bi-directional LSTM model (e.g., an Embeddings from Language Model (ELMo)) or a transformer model. In embodiments where the bi-directional contextual word embeddings model includes, or is based on, a transformer model, the transformer model may include decoder blocks (e.g., a Generative Pre-trained Transformer 2 (GPT-2) model), encoder blocks (e.g., a Bidirectional Encoder Representations from Transformers (BERT) model) and/or encoder/decoder architecture (a T5 or GPT-3 model). In some embodiments, the machine learning model includes, or is based on, a masked-language model such as a BERT model.

As discussed above, the machine learning model may be a bi-directional contextual word embeddings model (e.g., BERT). In these instances, the machine learning model may be pre-trained. By pre-trained is meant the machine learning model is first trained on one task or dataset before training the pre-trained model on another task or dataset. In embodiments of the methods, pre-training occurs before training using the clinical notes annotated as discussed above. In some embodiments, the machine learning model may be pre-trained on a large corpus of text such as, e.g., a large corpus of text relevant or associated with the tasks the machine learning model is trained to perform. For example, the machine learning model may be pre-trained using a large corpus of EHR data such as, e.g., a large corpus of PHI redacted clinical notes. In some embodiments, the pre-training may be unsupervised or self-supervised (i.e., desired outputs or classifications are not provided for pre-training). In some cases, the machine learning model may be configured to process inputs of a specific size such as, e.g., a size compatible with the inputs used to pre-train and/or train the machine learning model and/or the tasks performed by the machine learning model (e.g., inferring relationships between words or phrases in a document or note of a specific size). In these instances, the machine learning model may be hierarchical, e.g., the machine learning model may combine inputs or deal with inputs hierarchically. For example, in embodiments where the machine learning model includes, or is based on, a BERT model, the BERT based machine learning model may include one or more additional transformer layers, such as two or more layers, or three or more, or five or more, or ten or more. In these instances, the hierarchical BERT model may be configured to encode sequences of 1024 tokens or greater, or 2048 tokens or greater, or 2500 tokens or greater, or 2560 tokens or greater.

The machine learning model may be trained to perform any of the tasks demonstrated, or enabled, by the annotations as described above. For example, the machine learning model may be trained to perform one or more of the tasks (or, e.g., subtasks when combined as discussed above) of: indicating or inferring a relationship between a medical intervention and a context of interest, indicating or inferring a relationship between an AE and a context of interest, indicating or inferring a relationship between an AE and a medical intervention of interest, etc. In some embodiments, the machine learning model may be trained to perform any task associated with pharmacovigilance including, but not limited to, identifying adverse events associated with a medical intervention, identifying notes based on the presence or absence of at least one documented SAE, identifying whether a medical intervention such as, e.g., a medication was documented as being regularly given at the time of a mentioned hospitalization, identifying whether a disease/condition diagnosis corresponded to the primary reason for a hospitalization, identifying whether or not a given triplet of documented medical intervention (e.g., medication), hospitalization, and diagnosis corresponded to a valid SAE, etc. by performing one or more tasks/subtasks enabled by clinical notes annotated as discussed above.

Training and pre-training may depend on the nature or architecture of the machine learning model. For example, in embodiments where the machine learning model includes, or is based on, BERT architecture, training and/or pre-training may include masked language modeling, next sentence prediction, or similar variations thereof. In some embodiments, placeholders or tokens may be used to train the machine learning model. For example, in embodiments where all medical interventions and all AEs are labeled, all medical interventions (including, e.g., medical interventions of interest) may be replaced with a general medical intervention token and all AEs (including, e.g., AEs of interest) may be replaced with a general AE token. In some embodiments, pre-training may be unsupervised or self-supervised and training may be supervised using the clinical notes annotated as discussed above. In some embodiments, the model training algorithms and hyperparameters used to control the training may depend on, e.g., the nature or architecture of the machine learning model, the tasks the machine learning model is trained to perform, the desired accuracy or efficiency of the machine learning model, and/or the nature or size of the training data set.

In some cases, the training and/or the training data set (i.e., the annotated clinical notes) may be modified or altered to address class imbalance. By class imbalance is meant a skewed proportion of the classes that make up a data set. For example, annotated clinical notes reflecting a specific relationship or classification (e.g., annotated clinical notes including SAEs or AEs related or associated with a medical intervention of interest) may be relatively uncommon in the data set. In some embodiments, the training may be modified or altered to address class imbalance. For example, the optimization loss may be weighted based on class distributions. In these cases, the weighting may be learned dynamically, e.g., during training. In some embodiments, the training data set (i.e., the annotated clinical notes) may be modified to address class imbalance. In these instances, the majority class may be undersampled. For example, in embodiments where annotated clinical notes including AEs related or associated with a medical intervention of interest are relatively rare, annotated clinical notes without AEs related or associated with a medical intervention of interest may be randomly undersampled. In some embodiments, the majority class or classes may be randomly undersampled to achieve a ratio of one to five minority class (i.e., rare relationship or classification) to majority class(es) or less. In some instances, the majority class(es) may be undersampled to achieve a ratio of one to fifty minority class (i.e., rare relationship or classification) to majority class(es) or less, such one to twenty, or one to ten, or one to five, or one to four.

In some embodiments, the training may further include testing the trained machine learning model or machine learning models. By testing in this context is meant evaluating the trained machine learning model using annotated clinical notes different from the annotated clinical notes used for training after the machine learning model has finished training. In some embodiments, a first subset of the plurality of annotated clinical notes is used for training and a second subset of the plurality of annotated clinical notes is used for testing. The testing may use one or more metrics to evaluate the performance of the trained machine learning model or machine learning models. In some cases, the metric may include the number, or percent, of true positives, false positives, true negatives, or false negatives for one or more classes. In some embodiments, the metric may include a sensitivity, specificity, accuracy and/or f-score. In some instances, a metric may be determined per class. In embodiments where the metric includes an f-score, the f-score may include a macro F1-score. In some embodiments, the metric may be used to determine if the trained machine learning model performs sufficiently using, e.g., a predetermined threshold (i.e., requirement). In these instances, if the trained machine learning model does not meet the predetermined threshold, the model may be discarded and/or another model may be trained. In embodiments where another machine learning model is trained, one or more of the model architecture, training and/or the training data set may be modified prior to training. In some instances, machine learning models are trained until a trained machine learning models meets the predetermined threshold. The division between the first and second subsets of the plurality of annotated clinical notes used for training and testing, respectively, may vary. In some cases, roughly 80% of the annotated clinical notes may be used for training and roughly 20% for testing. In some instances, roughly 70% of the annotated clinical notes may be used for training and roughly 30% for testing.

In some embodiments, the training may further include validating the trained machine learning model or machine learning models. By validating in this context is meant evaluating the machine learning model during training using annotated clinical notes different from the annotated clinical notes used for training and testing. In some embodiments, a first subset of the plurality of annotated clinical notes is used for training, a second subset of the plurality of annotated clinical notes is used for testing, and a third subset of the plurality of annotated clinical notes is used for validating. The validating may use one or more metrics to evaluate the performance of the machine learning model or machine learning models such as, e.g., any of the metrics discussed above for testing. In some embodiments, the validating may be used to, e.g., select model parameters (e.g., select one or more machine learning algorithms to continue training), optimize or tune hyperparameters (e.g., model hyperparameters or algorithm hyperparameters), etc. The division between the first, second, and third subsets of the plurality of annotated clinical notes used for training, testing, and validating, respectively, may vary. In some cases, roughly 80% of the annotated clinical notes may be used for training, roughly 10% for testing, and roughly 10% for validating.

As discussed above, embodiments of the methods include training a machine learning model to identify adverse events associated with a medical intervention using the annotated clinical notes (e.g., annotated as described above). The machine learning model may be a deep learning model configured to process sequential input data. In some embodiments, the machine learning model is bi-directional contextual word embeddings model such as BERT. In these instances, the BERT model may be pre-trained using a large corpus of EHR data and may be hierarchical. The machine learning model may be trained to perform any task associated with pharmacovigilance including any of the tasks demonstrated, or enabled, by the annotations as described above. In some embodiments, the training data set (i.e., the annotated clinical notes) may be modified to address class imbalance. For example, minority classes may be randomly undersampled during training to achieve a ratio of roughly one to four minority class to majority class. In some embodiments, the training may further include validating the machine learning model during training and during the trained machine learning model after training. In some embodiments, a first subset of the plurality of annotated clinical notes is used for training, a second subset of the plurality of annotated clinical notes is used for testing, and a third subset of the plurality of annotated clinical notes is used for validating. The trained machine learning model may be applied to a second subset of the preprocessed clinical notes to perform one or more tasks, as discussed in greater detail below.

### Applying the Machine Learning Model

Embodiments of the methods include applying the trained machine learning model to a second subset of the preprocessed clinical notes to perform one or more tasks. By applying is meant to implement or use the trained machine learning model or, e.g., inputting or feeding the second subset of the preprocessed clinical notes to the trained machine learning model such that the trained machine learning model performs one or more tasks, such as any of the tasks described above. In some embodiments, the trained machine learning model may be applied to the second subset of the preprocessed clinical notes to identify adverse effects associated with a medical intervention. The applying may include extracting information using the machine learning model to draw one or more conclusions or to perform any task associated with pharmacovigilance. In some embodiments, the applying may further include generalizing or normalizing one or more of the identified adverse effects associated with the medical intervention. The applying may further includes compiling the results of the one or more tasks performed by the trained machine learning model and transmitting the results for further analysis or use.

As described above, the trained machine learning model may be applied to the second subset of the preprocessed clinical notes to draw one or more conclusions or to perform any task associated with pharmacovigilance such as, e.g., any of the tasks described above. In some embodiments, the trained machine learning model may be applied to infer a relationship. For example, the trained machine learning model may be applied to infer a relationship between an AE and a context of interest, a relationship between a medical intervention and a context of interest, a relationship between an AE and a medical intervention of interest, and/or a relationship between a disease or condition of interest (e.g., a disease or condition diagnosed in a cohort of interest) and an AE of interest.

In some embodiments, the trained machine learning model may be applied for identification or classification. For example, the trained machine learning model may be applied to identify all events of interest (e.g., hospitalizations, deaths, births, etc.) occurring as the result of an AE of interest, all medical interventions occurring before events of interest and during a relevant time window (e.g., a time window determined to be compatible with a causal relationship between an AE and a medical intervention before an individual has stopped receiving, or being exposed to, the medical intervention), all AEs associated with a medical intervention, all clinical notes with the presence of at least one documented SAE, all instances when a medical intervention such as, e.g., a medication was documented as being regularly given at the time of a mentioned hospitalization, all instances when a disease/condition diagnosis corresponded to the primary reason for a hospitalization, and/or all instances when a given triplet of documented medical intervention (e.g., medication), hospitalization, and diagnosis corresponded to a valid SAE.

The trained machine learning model may be applied to the second subset of the preprocessed clinical notes through any number of various means. In some embodiments, manually define, rule-based classifiers may be implemented with the trained machine learning model to determine or infer any of the relationships as described above. In some embodiments, one or more parameters may be adjusted when prompting the trained machine learning model to perform one or more tasks. In some instances, relationships may be restricted to those occurring between components appearing within a certain number of words or sentences. For example, the trained machine learning model may be prompted to identify hospitalizations related to SAEs while restricting to SAEs mentioned within two sentence spans from the hospitalization. In some embodiments, the trained machine learning model may be applied the second subset of the preprocessed clinical notes automatically using, e.g., lines of computer code.

In some embodiments, the applying may further include generalizing or normalizing one or more of the identified or classified components. In some cases, AEs (e.g., SAEs) identified for any reason described above may be normalized. In some embodiments, one or more of the identified or classified components may be normalized to a controlled vocabulary such as, e.g., the controlled vocabulary of a particular database or system. For example, in embodiments where the components are SAEs, the SAEs may be normalized using the controlled vocabulary of the UMLS metathesaurus or MedDRA. In some embodiments, the normalizing may be performed automatically using rules-based approaches such as, e.g., rules-based approaches implemented using lines of computer code. In some instances, the normalizing may be performed using supervised machine learning.

In some embodiments, the applying may further include aggregating the information obtained using the machine learning model, e.g., as described above. In these instances, the aggregated data may be used to generate a report. In some embodiments, the aggregated information may be transmitted or reported for downstream analysis. For example, in embodiments where the trained machine learning model identifies potential medical intervention related AEs, the clinical notes identifying the AEs may be reported or transmitted for causal analysis.

As discussed above, embodiments of the methods include applying the trained machine learning model to a second subset of the preprocessed clinical notes to perform one or more tasks. The trained machine learning model may be applied to the second subset of the preprocessed clinical notes to draw one or more conclusions or to perform any task associated with pharmacovigilance such as, e.g., any of the tasks described above. In some embodiments, the trained machine learning model identifies potential medical intervention related AEs. The trained machine learning model may be applied to the second subset of the preprocessed clinical notes through any number of various means. In some embodiments, one or more parameters may be adjusted when prompting the trained machine learning model to perform one or more tasks. For example, relationships may be restricted to those occurring between components appearing within a certain number of words or sentences. In some embodiments, the applying may further include generalizing or normalizing one or more of the identified or classified components such as, e.g., normalizing identified AEs using the controlled vocabulary of the UMLS metathesaurus or MedDRA. In some embodiments, the applying may further include aggregating and reporting the information obtained using the machine learning model for downstream analysis.

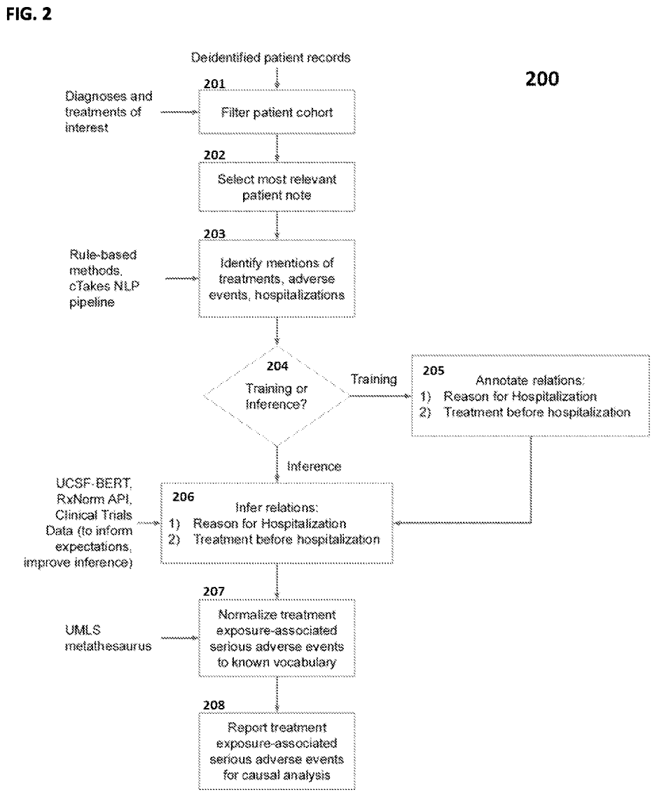

FIG. 2 provides a flow diagram depicting method 200 of identifying medical intervention related AEs from EHR data in accordance with an embodiment of the invention. At step 201, a plurality of clinical notes is obtained by filtering, e.g., a large corpus of PHI redacted clinical notes to include only the clinical notes pertaining to a cohort of interest. The cohort of interest may include patients diagnosed with a specific disease or condition of interest and/or receiving a specific treatment of interest. At step 202, the obtained plurality of clinical notes is further reduced to only include the most relevant patient note such as, e.g., the most recent clinical note during a window after a patient has started and before the patient has stopped receiving the treatment of interest. At step 203, rules-based methods are applied to identify and, e.g., label treatments, AEs, and hospitalizations. The identifying and labeling may be performed, at least in part, using an NLP pipeline such as an NLP pipeline including cTAKES. At step 204, labeled clinical notes are divided into two subsets of notes. The first subset is annotated at step 205 to indicate the reason for each mention hospitalization and occurrences where a hospitalization occurred after a treatment. A machine learning model, such as UCSF-BERT, is trained using the annotated clinical notes and applied to the second subset of labeled clinical notes at step 206 to infer relationships in the unannotated notes. The trained machine learning model, the RxNorm application programming interface (API), and data from clinical trials may all be used to identify the reason for each mention hospitalization and occurrences where a hospitalization occurred after a treatment in order to identify treatment exposure associated SAEs. In some cases, the clinical trial data may be used to inform expectations and improve inferences. For example, identified treatment exposure associated SAEs may be verified if they have been previously identified in clinical trials data, while identified treatment exposure associated SAEs absent from clinical trials data may be verified by downstream causal analysis. At step 207, identified treatment exposure associated SAEs may be normalized to a controlled vocabulary using UMLS metathesaurus and, e.g., automatically implemented rules-based methods. At step 207, identified treatment exposure associated SAEs may be aggregated and reported for causal analysis.

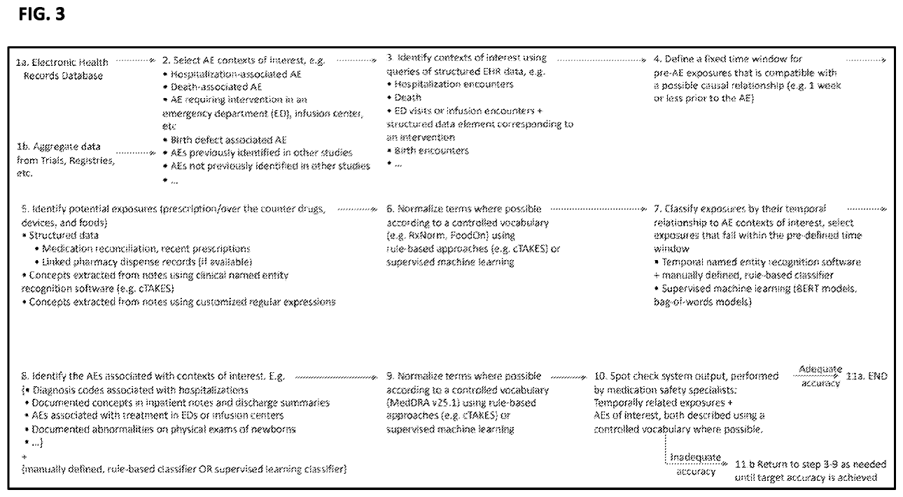

FIG. 3 provides an overview of a method of identifying medical intervention related AEs from EHR data in accordance with an embodiment of the invention. At step 1a, an EHR database is used to obtained a plurality of clinical notes and at step 1b data pertaining to, e.g., medical interventions of interest such as specific mediations is aggregated from trials (e.g., clinical trials) and registries. At step 2, AE contexts of interest are selected. In some cases, the EHR data and the data aggregated from the trials and registries may be used to inform the AE context selection. At step 3, the contexts of interest may be identified using queries of structured EHR data and, e.g., rules based approaches. At step 4, a fixed window of time is defined concerning how far before an AE a medical intervention has to occur to be compatible with a potential causal relationship. In some cases, a window may also be defined concerning how long after an exposure to a medical intervention an AE may occur for it to be compatible with a potential causal relationship. At step 5, potential exposures (e.g., to medical inventions such as specific treatments) are identified and, e.g., labeled using structured data and clinical name recognition. At step 6, identified medical intervention exposures (e.g., medication or food consumption) may be normalized using, e.g., RxNorm or FoodON and automatically implemented rules-based approaches or supervised machine learning. At step 7, exposures are classified by their temporal relationship to an AE context of interest (e.g., a hospitalization or death) and exposures that fall within the pre-defined time window of step 4 are selected. The classification and selection may occur using a supervised machine learning model such as BERT or a BoW model, temporal named entity recognition software, and a manually defined rule-based classifier. At step 8, AEs associated with the contexts of interest are identified. The identification may occur using a manually defined rule-based classifier or a supervised learning classifier, e.g., a supervised machine learning model such as BERT. At step 9, terms such as identified treatment exposure associated AEs may be normalized to a controlled vocabulary (e.g., MedDRA) using, e.g., automatically implemented rules-based approaches or supervised machine learning. At step 10, the system output is spot checked by medication safety specialists. At step 11a, if the output has inadequate accuracy, step 3 through step 9 are repeated until target accuracy is achieved. Once target accuracy has been achieved, the method ends at step 11b.

### Computer Implemented Embodiments

The various method steps described in connection with the embodiments disclosed herein can be implemented as electronic hardware, computer software, or combinations of both. To clearly illustrate this interchangeability of hardware and software, various illustrative steps have been described above generally in terms of their functionality. Whether such functionality is implemented as hardware or software depends upon the particular application and design constraints imposed on the overall system applying a method according to the present disclosure. The described functionality can be implemented in varying ways for each particular application, but such implementation decisions should not be interpreted as causing a departure from the scope of the disclosure.

The various illustrative steps, components, and computing systems (such as devices, databases, interfaces, and engines) described in connection with the embodiments disclosed herein can be implemented or performed by a machine, such as a general purpose processor, a graphics processor unit, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor can be a microprocessor, but in the alternative, the processor can be a controller, microcontroller, or state machine, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor can also include primarily analog components. A computing environment can include any type of computer system, including, but not limited to, a computer system based on a microprocessor, a graphics processor unit, a mainframe computer, a digital signal processor, a portable computing device, a personal organizer, a device controller, and a computational engine within an appliance, to name a few.

The steps of a method, process, or algorithm described in connection with the embodiments disclosed herein can be embodied directly in hardware, in a software module executed by a processor, or in a combination of the two. A software module, engine, and associated databases can reside in memory resources such as in RAM memory, FRAM memory, flash memory, ROM memory, EPROM memory, EEPROM memory, registers, hard disk, a removable disk, a CD-ROM, or any other form of non-transitory computer-readable storage medium, media, or physical computer storage known in the art. An external storage medium can be coupled to the processor such that the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

As described in detail above, embodiments of the present invention relate to methods of identifying medical intervention related adverse events (AEs) from electronic health records (EHR) data. Aspects of the methods include: obtaining a plurality of health records including clinical notes regarding individuals associated with the medical intervention; preprocessing the plurality of clinical notes to include medical intervention and adverse event labels; annotating a first subset of the preprocessed clinical notes to indicate if each labeled adverse event is associated with the medical intervention; training a machine learning model to identify adverse events associated with the medical intervention using the annotated clinical notes; and applying the trained machine learning model to a second subset of the preprocessed clinical notes to identify adverse events associated with the medical intervention. Aspects of the present invention further include methods of identifying contexts of AEs indicative of the nature or severity of AEs of interest.

### Systems

Aspects of the present disclosure further include systems, such as computer-controlled systems, for practicing embodiments of the above methods. Aspects of the systems include: a processor including memory operably coupled to the processor, wherein the memory includes instructions stored thereon, which, when executed by the processor, cause the processor to: obtain a plurality of health records including clinical notes regarding individuals associated with the medical intervention; preprocess the plurality of clinical notes to include medical intervention and adverse effect labels; annotate a first subset of the preprocessed clinical notes to indicate if each labeled adverse effect is associated with the medical intervention; train a machine learning model to identify adverse effects associated with the medical intervention using the annotated first subset of clinical notes; apply the trained machine learning model to a second subset of the preprocessed clinical notes to identify adverse effects associated with the medical intervention. The systems allow for adverse effects associated with the medical intervention to be identified from clinical notes, as discussed above.

In some embodiments, the machine learning model may include an artificial neural network (NN) (e.g., a convolutional NN (CNN)). In some embodiments, the machine learning model is a deep learning model. In these cases, the model may be three or more layers deep, such as five or more layers deep, or ten or more, or twelve or more, or sixteen or more, or twenty-four or more, or thirty-two or more, or sixty-four or more. In some embodiments, the machine learning model is configured to process sequential input data. In these instances, the machine learning model may include, or be based on, a recurrent neural network (RNN) model or a transformer model. In embodiments where the machine learning model includes an RNN, the RNN may include, e.g., long short-term memory (LSTM) architecture and/or gated recurrent units (GRUs). In some embodiments, the machine learning model may include, or be based on, the architecture of a transformer model.

As discussed above, the machine learning model may be configured to process sequential input data. The sequential input data may be a sequence of words, and the machine learning model may be configured to use the bag-of-words (BoW) model and/or word embeddings to represent the sequence of words. In embodiments using the BoW model, the machine learning model may include a Logistic Regression, K-Nearest Neighbors, Decision Tree, Random Forest and/or XGBoost model. In some embodiments, the machine learning model may use word embeddings to represent the sequence of words. In these instances, the machine learning model may be configured to learn from the contextual information of a word (i.e., the words before or after a given word sequentially). The machine learning model may learn from the contextual information of a word using contextual word embeddings and, e.g., may learn from the left to right context of a word and/or the right to left context of a word. In some embodiments, the machine learning model may learn from both the left to right context and the right to left context of a word (i.e., the machine learning model may be bidirectional). For example, the machine learning model may include, or be based on, a bi-directional LSTM model (e.g., an Embeddings from Language Model (ELMo)) or a transformer model. In embodiments where the bi-directional contextual word embeddings model includes, or is based on, a transformer model, the transformer model may include decoder blocks (e.g., a Generative Pre-trained Transformer 2 (GPT-2) model), encoder blocks (e.g., a Bidirectional Encoder Representations from Transformers (BERT) model) and/or encoder/decoder architecture (a T5 or GPT-3 model). In some embodiments, the machine learning model includes, or is based on, a masked-language model such as a BERT model.

As discussed above, the machine learning model may be a bi-directional contextual word embeddings model (e.g., BERT). In these instances, the machine learning model may be pre-trained. By pre-trained is meant the machine learning model is first trained on one task or dataset before training the pre-trained model on another task or dataset. In embodiments of the methods, pre-training occurs before training using the clinical notes annotated as discussed above. In some embodiments, the machine learning model may be pre-trained on a large corpus of text such as, e.g., a large corpus of text relevant or associated with the tasks the machine learning model is trained to perform. For example, the machine learning model may be pre-trained using a large corpus of EHR data such as, e.g., a large corpus of PHI redacted clinical notes. In some embodiments, the pre-training may be unsupervised or self-supervised (i.e., desired outputs or classifications are not provided for pre-training). In some cases, the machine learning model may be configured to process inputs of a specific size such as, e.g., a size compatible with the inputs used to pre-train and/or train the machine learning model and/or the tasks performed by the machine learning model (e.g., inferring relationships between words or phrases in a document or note of a specific size). In these instances, the machine learning model may be hierarchical, e.g., the machine learning model may combine inputs or deal with inputs hierarchically. For example, in embodiments where the machine learning model includes, or is based on, a BERT model, the BERT based machine learning model may include one or more additional transformer layers, such as two or more layers, or three or more, or five or more, or ten or more. In these instances, the hierarchical BERT model may be configured to encode sequences of 1024 tokens or greater, or 2048 tokens or greater, or 2500 tokens or greater, or 2560 tokens or greater.

In some embodiments, the memory includes instructions stored thereon, which when executed by the processor, further cause the processor to annotate a first subset of the preprocessed clinical notes to indicate if each labeled adverse effect is associated with the medical intervention based on the input provided by one or more annotators. In some embodiments, the memory includes instructions stored thereon, which when executed by the processor, further cause the processor to prompt or instruct the one or more annotators to provide annotation input. In some embodiments, the system may further include means for the one or more annotators to provide input to the processor. In some cases, the input means includes one or more operator input devices. Operator input devices may, for example, be a touchscreen, a keyboard, a mouse, or the like. In some embodiments, the system may further include means to prompt or instruct the one or more annotators to provide annotation input. In some cases, the instruction means includes one or more visual displays. The displays may include electronic display devices such as, e.g., a liquid crystal display (LCD), an organic light-emitting diode (OLED) display or an active-matrix organic light-emitting diode (AMOLED) display. In some embodiments, the display device may include a projector. In some cases, the display includes a loudspeaker configured to provide audio instructions to the annotator.

In some embodiments, the memory includes instructions stored thereon, which when executed by the processor, further cause the processor to normalize the wording of the identified adverse effects associated with the medical intervention. In some embodiments, the memory includes instructions stored thereon, which when executed by the processor, further cause the processor to aggregate information output by the machine learning model and generate a report.

In some embodiments, the memory includes instructions stored thereon, which when executed by the processor, further cause the processor to train the machine learning model with a first subset of the annotated clinical notes and test the trained machine learning model using a second subset of the annotated clinical notes according to any of the methods as discussed above. In some embodiments, the memory includes instructions stored thereon, which when executed by the processor, further cause the processor to train the machine learning model with a first subset of the annotated clinical notes, test the trained machine learning model using a second subset of the annotated clinical notes, and validate the machine learning model during training using a third subset of the annotated clinical notes according to any of the methods as discussed above.

In some instances the systems further include one or more computers for complete automation or partial automation of the methods described herein. In some embodiments, systems include a computer having a computer readable storage medium with a computer program stored thereon.

In embodiments, the system includes an input module, a processing module and an output module. The subject systems may include both hardware and software components, where the hardware components may take the form of one or more platforms, e.g., in the form of servers, such that the functional elements, i.e., those elements of the system that carry out specific tasks (such as managing input and output of information, processing information, etc.) of the system may be carried out by the execution of software applications on and across the one or more computer platforms represented of the system.

The processing module includes a processor which has access to a memory having instructions stored thereon for performing the steps of the subject methods. The processing module may include an operating system, a graphical user interface (GUI) controller, a system memory, memory storage devices, and input-output controllers, cache memory, a data backup unit, and many other devices. The processor may be a commercially available processor or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as Java, Perl, C++, Python, other high-level or low-level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques. The processor may be any suitable analog or digital system. In some embodiments, the processor includes analog electronics which provide feedback control, such as for example negative feedback control.

The system memory may be any of a variety of known or future memory storage devices. Examples include any commonly available random access memory (RAM), magnetic medium such as a resident hard disk or tape, an optical medium such as a read and write compact disc, flash memory devices, or other memory storage device. The memory storage device may be any of a variety of known or future devices, including a compact disk drive, a tape drive, a removable hard disk drive, or a diskette drive. Such types of memory storage devices typically read from, and/or write to, a program storage medium (not shown) such as, respectively, a compact disk, magnetic tape, removable hard disk, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with the memory storage device.

In some embodiments, a computer program product is described including a computer usable medium having control logic (computer software program, including program code) stored therein. The control logic, when executed by the processor the computer, causes the processor to perform functions described herein. In other embodiments, some functions are implemented primarily in hardware using, for example, a hardware state machine.

Implementation of the hardware state machine so as to perform the functions described herein will be apparent to those skilled in the relevant arts.

Memory may be any suitable device in which the processor can store and retrieve data, such as magnetic, optical, or solid-state storage devices (including magnetic or optical disks or tape or RAM, or any other suitable device, either fixed or portable). The processor may include a general-purpose digital microprocessor suitably programmed from a computer readable medium carrying necessary program code. Programming can be provided remotely to processor through a communication channel, or previously saved in a computer program product such as memory or some other portable or fixed computer readable storage medium using any of those devices in connection with memory. For example, a magnetic or optical disk may carry the programming, and can be read by a disk writer/reader. Systems of the invention also include programming, e.g., in the form of computer program products, algorithms for use in practicing the methods as described above. Programming according to the present invention can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; portable flash drive; and hybrids of these categories such as magnetic/optical storage media.

The processor may also have access to a communication channel to communicate with a user at a remote location. By remote location is meant the user is not directly in contact with the system and relays input information to an input manager from an external device, such as a computer connected to a Wide Area Network (“WAN”), telephone network, satellite network, or any other suitable communication channel, including a mobile telephone (i.e., smartphone).

In some embodiments, systems according to the present disclosure may be configured to include a communication interface. In some embodiments, the communication interface includes a receiver and/or transmitter for communicating with a network and/or another device. The communication interface can be configured for wired or wireless communication, including, but not limited to, radio frequency (RF) communication (e.g., Radio-Frequency Identification (RFID), Zigbee communication protocols, WiFi, infrared, wireless Universal Serial Bus (USB), Ultra Wide Band (UWB), Bluetooth® communication protocols, and cellular communication, such as code division multiple access (CDMA) or Global System for Mobile communications (GSM).

In one embodiment, the communication interface is configured to include one or more communication ports, e.g., physical ports or interfaces such as a USB port, an RS-232 port, or any other suitable electrical connection port to allow data communication between the subject systems and other external devices such as a computer terminal (for example, at a physician's office or in hospital environment) that is configured for similar complementary data communication.

In one embodiment, the communication interface is configured for infrared communication, Bluetooth® communication, or any other suitable wireless communication protocol to enable the subject systems to communicate with other devices such as computer terminals and/or networks, communication enabled mobile telephones, personal digital assistants, or any other communication devices which the user may use in conjunction.

In one embodiment, the communication interface is configured to provide a connection for data transfer utilizing Internet Protocol (IP) through a cell phone network, Short Message Service (SMS), wireless connection to a personal computer (PC) on a Local Area Network (LAN) which is connected to the internet, or WiFi connection to the internet at a WiFi hotspot.

In one embodiment, the subject systems are configured to wirelessly communicate with a server device via the communication interface, e.g., using a common standard such as 802.11 or Bluetooth® RF protocol, or an IrDA infrared protocol. The server device may be another portable device, such as a smart phone, Personal Digital Assistant (PDA) or notebook computer; or a larger device such as a desktop computer, appliance, etc. In some embodiments, the server device has a display, such as a liquid crystal display (LCD), as well as an input device, such as buttons, a keyboard, mouse or touch-screen.

In some embodiments, the communication interface is configured to automatically or semi-automatically communicate data stored in the subject systems, e.g., in an optional data storage unit, with a network or server device using one or more of the communication protocols and/or mechanisms described above.

Output controllers may include controllers for any of a variety of known display devices for presenting information to a user, whether a human or a machine, whether local or remote. If one of the display devices provides visual information, this information typically may be logically and/or physically organized as an array of picture elements. A graphical user interface (GUI) controller may include any of a variety of known or future software programs for providing graphical input and output interfaces between the system and a user, and for processing user inputs. The functional elements of the computer may communicate with each other via system bus. Some of these communications may be accomplished in alternative embodiments using network or other types of remote communications. The output manager may also provide information generated by the processing module to a user at a remote location, e.g., over the Internet, phone or satellite network, in accordance with known techniques. The presentation of data by the output manager may be implemented in accordance with a variety of known techniques. As some examples, data may include SQL, HTML or XML documents, email or other files, or data in other forms. The data may include Internet URL addresses so that a user may retrieve additional SQL, HTML, XML, or other documents or data from remote sources. The one or more platforms present in the subject systems may be any type of known computer platform or a type to be developed in the future, although they typically will be of a class of computer commonly referred to as servers. However, they may also be a main-frame computer, a workstation, or other computer type. They may be connected via any known or future type of cabling or other communication system including wireless systems, either networked or otherwise. They may be co-located or they may be physically separated. Various operating systems may be employed on any of the computer platforms, possibly depending on the type and/or make of computer platform chosen. Appropriate operating systems include Windows, iOS, Oracle Solaris, Linux, IBM i, Unix, and others.

Aspects of the present disclosure further include non-transitory computer readable storage mediums having instructions for practicing the subject methods. Computer readable storage mediums may be employed on one or more computers for complete automation or partial automation of a system for practicing methods described herein. In certain embodiments, instructions in accordance with the method described herein can be coded onto a computer-readable medium in the form of “programming”, where the term “computer readable medium” as used herein refers to any non-transitory storage medium that participates in providing instructions and data to a computer for execution and processing. Non-transitory computer-readable media include all computer-readable media except for a transitory, propagating signal. Examples of suitable non-transitory storage media include a floppy disk, hard disk, optical disk, magneto-optical disk, CD-ROM, CD-R, magnetic tape, non-volatile memory card, ROM, DVD-ROM, Blue-ray disk, solid state disk, and network attached storage (NAS), whether or not such devices are internal or external to the computer. A file containing information can be “stored” on computer readable medium, where “storing” means recording information such that it is accessible and retrievable at a later date by a computer. The computer-implemented method described herein can be executed using programming that can be written in one or more of any number of computer programming languages. Such languages include, for example, Python, Java, Java Script, C, C#, C++, Go, R, Swift, PHP, as well as many others.

The non-transitory computer readable storage medium may be employed on one or more computer systems having a display and operator input device. Operator input devices may, for example, be a keyboard, mouse, or the like. The processing module includes a processor which has access to a memory having instructions stored thereon for performing the steps of the subject methods. The processing module may include an operating system, a graphical user interface (GUI) controller, a system memory, memory storage devices, and input-output controllers, cache memory, a data backup unit, and many other devices. The processor may be a commercially available processor or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as those mentioned above, other high level or low level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques.

### Utility

The methods and systems of the invention, e.g., as described above, find use in a variety of applications where it is desirable to accurately and cost effectively monitor the safety of various medical interventions such as the safety of drugs and medical devices. In some embodiments, the methods and systems described herein find use when it is desirable to reduce the manual efforts needed to review clinical notes and identify events of concern. In some embodiments, the methods and systems described herein find use when it is desirable to reduce the costs associated with treatment exposure-related adverse events. Embodiments of the present disclosure find use in applications wherein it is desired to monitor and report safety-related events to governmental bodies that are charged with ensuring the safety of marketed drugs and medical devices (e.g. US Food and Drug Administration, European Medicines Agency). In some embodiments, methods and systems described herein find use when it is desirable to characterize or validate a medical intervention safety profile and/or identify previously unrecognized AEs or SAEs associated with a medical intervention. In some embodiments, the subject methods and systems may facilitate the research and development of various drugs, medical devices, and other medical interventions.

### EXPERIMENTAL

As demonstrated in the above disclosure, the present invention has a wide variety of applications. The following examples are put forth so as to provide those of ordinary skill in the art with a complete disclosure and description of how to make and use the present invention and are not intended to limit the scope of what the inventors regard as their invention nor are they intended to represent that the experiments below are all or the only experiments performed. Those of skill in the art will readily recognize a variety of noncritical parameters that could be changed or modified to yield essentially similar results. Efforts have been made to ensure accuracy with respect to numbers used (e.g. amounts, percentages, etc.) but some experimental errors and deviations should be accounted for.

### Introduction

Described herein are experiments and examples wherein embodiments of the present invention are applied in the context of steroid-sparing immunosuppressants for inflammatory bowel disease (IBD), as documented in outpatient clinical notes. Methods and evaluations of several embodiments of the invention adapted to perform different tasks related to hospitalization-associated serious adverse events (SAE) detection are reported, and the embodiments of the invention were compared to a range of baselines including published state-of-the-art models.

The target of prediction for the following experiments and examples are treatment-related SAEs associated with exposure to steroid-sparing immunosuppressant drugs for IBD, as documented in the outpatient gastroenterology clinic at UCSF. The candidate list of drugs included all biologics and small-molecule medications that were approved by the FDA for the treatment of Ulcerative colitis or Crohn's disease as of 2020, as well as off-label medications that are occasionally used to treat these conditions. Although the FDA's definition of SAEs includes multiple categories, only SAEs associated with a hospitalization event were explored. This is because hospitalizations are a clinically important event that is more likely to be documented well in clinical notes. However, the invention may be used to explore any documented SAE. Treatment related was defined as a SAE that occurred while the patient was actively receiving scheduled doses of a given medication. For example, if a patient was hospitalized 6 weeks after receiving an infusion that was prescribed to be given every 8 weeks, the hospitalization event would be considered as potentially corresponding to an SAE. Once the clinical decision to discontinue a given treatment plan was documented, subsequent hospitalization events were no longer considered associated with a given treatment.

### Example 1: Obtaining, Processing, and Annotating a Clinical Note Dataset

### Methods

### Obtaining Electronic Health Records

To identify target notes, a deidentified research database was used consisting of structured electronic health record (HER) data at UCSF as well as machine protected health information (PHI)-redacted clinical notes [1]. The database was queried to identify all notes associated with the gastroenterology department and an IBD diagnosis code (ICD-9 555/556; ICD-10 K50/K51). Notes written between Jan. 1, 2017 and Dec. 31, 2019 were selected, and the most recent note for each patient who was at least 18 years old during this period was utilized. This timeframe was selected to maximize the capture of a wide range of FDA-approved treatments and to limit potential biases related to the COVID-19 pandemic. Of note, as part of the deidentification process the true dates of these encounters were randomly shifted backwards in time by up to 365 days. The most recent note per patient was used to avoid double counting SAEs, and to take advantage of the fact the documented histories tend to be inclusive of prior events. All included notes were written by a gastroenterology physician or advanced practice provider in the outpatient clinic.

### Pre-Processing the Clinical Notes

The history of present illness (HPI) section of the notes was extracted using rule-based approaches that can be adjusted based on use (e.g., desired target of prediction and timeframe). The HPI section of the note was the only portion of the note utilized for downstream analysis as this section of the note is the most reliable and cumulative source of information on treatment exposures and outcomes, particularly out of system events (i.e. hospitalizations and SAEs that occurred outside of UCSF, but were relayed to the gastroenterology provider at the time of routine follow-up). The HPI was pre-labeled with medications of interest, hospitalization and signs and symptoms using named entity recognition functions from the clinical natural language processing software cTAKES [2], as well as regular expressions (i.e. the ability to locate pre-defined keywords). To minimize downstream algorithmic confusion in learning medication names, the brand and generic names of medications mentioned in notes were replaced with the generic name using the RxNorm API in the Unified Medical Language System (UMLS) [3].

### Annotation Protocol

All notes that met the above inclusion criteria were annotated to fine-tune a Bidirectional Encoder Representations from Transformers (BERT) model specifically designed to interpret clinical text as typically documented in EHRs (described in greater detail below), hereinafter referred to as UCSF BERT, on a variety of adverse event (AE) detection-related tasks and to evaluate its performance against comparator models. A team of five annotators, consisting of gastroenterologists, pharmacists, pharmacovigilance experts and patients, carried out all annotation related tasks. These included the development and finalization of an annotation protocol, participation in interrater reliability assessments, and annotation of all target notes. The annotation protocol was collectively developed and refined over the course of weekly team meetings utilizing an initial subset of notes. Using LabelStudio [4], an open-source annotation platform, annotators marked up the prelabelled HPI section of candidate notes according to triplets of medication mentions, hospitalization mentions, and AE mentions, if they corresponded to a serious adverse event as per the developed protocol (FIG. 1). These annotations became the basis of the subsequent efforts to train UCSF BERT and other models to automate this process.

All five annotators participated in an interrater reliability assessment on a sample of 19 notes. The results of these assessments were reviewed in weekly meetings to improve the protocol as well as annotator compliance to it. Further, if ambiguity was encountered annotators kept a list of notes to discuss as a group and adjudicate on a case-by-case basis at the weekly meetings. A Fleiss' kappa statistic was computed to characterize the interrater reliability on the final round of assessments. Following this training and assessment phrase, the protocol was locked and the remainder of the corpus was annotated.

FIG. 1 depicts an example of prediction tasks from a clinical note in the corpus. Medications of interest were pre-annotated in blue, hospitalizations in red and signs and symptoms in yellow. Annotators marked up the HPI section where medications of interest predated a hospitalization (green and blue arrow) and AE causing hospitalization (red arrow).

### MeDRA

All SAEs were manually coded using the medical dictionary for regulatory activities (MedDRA) version 23.0 [8].

### Results

### Source Corpus, Patient Population, and the SAE Dataset

From a deidentified dataset of 1.1 million machine redacted clinical notes at UCSF, we identified a total of 928 notes corresponding to adults with IBD who were seen during the 2017-2019 period. The patients in our study were 53% female with an average age of 45 years old. The most common race of patients was white, followed by 13% unknown or declined to answer. Characteristic of patients included in the note corpus are depicted in Table 1, below:

TABLE 1
Characteristic of Patients

| Patients (N = 928) | | Count (%) | |
|---|---|---|---|
| Sex | Female | 489 | (52.69) |
| | Male | 438 | (47.20) |
| | Nonbinary | 1 | (0.11) |
| Age | Mean | 44.50 | |
| | 18-40 | 460 | (49.57) |
| | 41-60 | 313 | (33.73) |
| | >60 | 155 | (16.70) |
| Ethnicity | Not Hispanic or Latino | 832 | (89.66) |
| | Hispanic or Latino | 83 | (8.94) |
| | Unknown/Declined | 13 | (1.40) |
| Race | White or Caucasian | 670 | (72.28) |
| | Asian | 78 | (8.41) |
| | Black or African American | 41 | (4.42) |
| | American Indian or Alaska Native | 9 | (0.97) |
| | Other Pacific Islander | 1 | (0.11) |
| | Other and Unknown/Declined | 129 | (13.9) |
| IBD Diagnosis | Crohn's disease (ICD Codes: 555.*, K50.*) | 735 | (79.20) |
| | Ulcerative colitis (ICD Code: 556.*, K51.*) | 625 | (67.35) |

All 928 notes were annotated, and interrater reliability testing was performed on a set of 19 notes to characterize the quality of the annotated dataset. The mean observed agreement between the five annotators was 93-99% in all types of annotations, as can be seen in Table 2 below.

TABLE 2
Inter-rater Reliability Assessment Results

| | | Observed | Chance | Fleiss' |
|---|---|---|---|---|
| Annotation | Type | Agreement | Agreement | Kappa |
| Concept | AE | 99% | 96% | 0.62 |
| Concept | Hospitalized | 93% | 53% | 0.84 |
| Concept | Medication | 95% | 78% | 0.77 |
| Relations | Medication before | 97% | 87% | 0.76 |
| | Hospitalized | | | |
| Relations | Hospitalized for AE | 99% | 98% | 0.62 |

A total of 703 SAEs were identified in the notes. Out of the 928 annotated notes, 411 documented at least one SAE (Table 3 and FIG. 4). The notes documenting an SAE tended to be longer than those without an SAE (p<0.001). Over 60% of SAEs in the corpus were associated with anti-tumor necrosis factor agents. Infliximab was associated with 179 SAEs, the most of any drug, followed closely by adalimumab (136 SAEs; Table 3). The most common SAE was failure of intended efficacy, 299, followed by infections, 94 (FIG. 4). Infectious SAEs were more common with anti-tumor necrosis factor agents. One cancer, sarcoma, occurred during infliximab therapy. However, serious adverse events were found for every organ system and every steroid sparing immunosuppressant. The complete list of SAEs mapped by clinical note terms and MedDRA terms can be found in FIG. 4. However, to enable a more user-friendly exploration of trends in the data, an interactive web application was developed, allowing a user to build and manipulate network graphs for any desired combination of drug/medication classes, medications, and AEs of interest.

TABLE 3
Distribution of Notes Containing a SAE

| | | Development | |
|---|---|---|---|
| | Train (%) | (%) | Test (%) |
| # Annotated notes | 741 (80) | 93 (10) | 93 (10) |
| SAEs present | 335 (82) | 37 (9) | 39 (9) |
| No SAEs | 406 (79) | 56 (11) | 54 (10) |

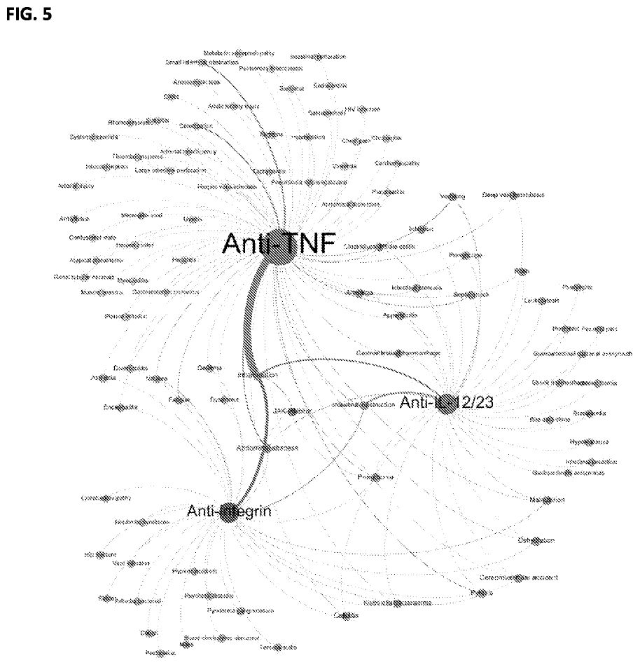

FIG. 5 depicts a Network Graph of SAEs by medication class. The width of lines indicates the strength of association by frequency. The size of the nodes is relative to the number of patients in our corpus exposed to each medication.

### Finalized Annotation Protocol

TABLE 4
Tasks Completed by Annotators Per Note

Task 1: Find a hospitalization that occurred AFTER starting a target
medication. Link all mentions of the same hospitalization together.
Task 2: Associate that hospitalization with every mention of the
target medication used prior to hospital admission
Task 3: Associate the hospitalization with the reason
for hospitalization

Task 1: Find hospitalizations that occur after starting medications of interest but before stopping the medications of interest. Link all mentions of the same hospitalization together.
1. Look at every hospitalization to determine if the hospitalization occurred AFTER starting a medication of interest
2. If a medication of interest was NOT started prior to the first hospitalization then move on to the next hospitalization until you find a hospitalization that occurs AFTER starting a medication of interest or all hospitalizations in the note have been reviewed and none occurred in the setting of medications of interest use.
3. The hospitalization must occur AFTER starting a medication of interest but BEFORE the decision is made to stop the medication of interest
4. If there are two or more words indicating the same hospitalization in the same or different sentences, they should be linked together in a path-connected fashion by relationship arrows. Once a hospitalization is found that occurred after the start of a medication of interest but before stopping the medication of interest and all assertions of this same hospitalization have been linked to each other proceed to Task 2.
   There may be multiple hospitalizations in a single note that occur after the start of a medication of interest. EVERY hospitalization that occurs AFTER a biologic has started but before stopping the biologic should be annotated.
   If a hospitalization is found that is not pre-annotated in red this can be done manually by clicking hospitalized and then using the cursor to highlight the word or phrase indicating hospitalization occurred.
Task 2: Link hospitalizations that occur after starting a medication of interest with the specific medication of interest used
1. Click on medication of interest name that is the medication of interest at time of hospitalization. If two medications of interest are being used concurrently prior to the hospitalization BOTH should be linked (this requires repeating this step individually for each biologic).
2. Click [symbol] icon on right hand side of screen. Also, a short cut can be used by holding down the “r” key on keyboard.
3. Click on hospitalization. A red bracket should appear between the medication of interest and hospitalization indicating the correct relationship has been made.
   a. The direction of the red arrow does not matter
   b. If the medication of interest is mentioned in the opening summary line of the note it should NOT be linked to the hospitalization.
4. Repeat this process for ALL mentions of the medication(s) of interest and link to the hospitalization(s).
   a. If there are multiple mentions of the same hospitalization, as long as all mentions of the same hospitalization are linked in a path connected fashion from Task 1 then all mentions of treatment-associated medication(s) of interest need only be linked to one mention of the hospitalization.
   b. If there are multiple treatment associated hospitalizations this process should be repeated for each.
Task 3: Link the hospitalization that occurred after biologic started with the reason for hospitalization. If no reason for hospitalization then DO NOT LABEL ANYTHING!
1. Click on the reason for hospitalization. This should be the highest-level diagnosis(s) that led to hospitalization and must occur within 2 sentences before or after the mention of the hospitalization. If the reason for hospitalization is mentioned multiple times within 2 sentences before or after hospitalization then the synonymous reasons for hospitalization should be connected to each other.
   a. If available, prefer more specific diagnosis (Ex. Prefer small bowel obstruction rather than obstruction. Ex prefer salmonella infections rather than infection.)
   b. If IBD (ulcerative colitis, Crohn's, colitis) is the reason for hospitalization then pick this. However, if “flare” is all that is used then you can highlight flare. Otherwise DO NOT include flare in the annotation.
   c. DO NOT include adjectives such as severe or acute in the annotation of reason for hospitalization.
   d. If no diagnosis is indicated, then it is acceptable to label symptom(s) as a reason(s) for hospitalization.
   e. If no diagnosis or symptoms are indicated as a reason for hospitalization then DO NOT annotate anything.
   f. Treatments or test results, such as surgeries, should not be labeled as reasons for hospitalization unless a diagnosis is stated from them (e.g., CT consistent with flare of colitis)
   g. If the reason for hospitalization is redacted, DO NOT label it as the reason for hospitalization.
2. Click [symbol] icon on right hand side of screen. Also, can use short cut with “r” key on keyboard.
3. Click on hospitalization. A red bracket should appear between the reason for hospitalization and hospitalization indicating the correct relationship has been made.
   a. The direction of the red arrow does not matter.

### Example 2: Developing a BERT Model for Clinical Note Analysis

### Methods

### Developing a BERT Model for Clinical Language

A BERT model was developed that was specifically designed to interpret clinical text as typically documented in electronic health records (EHR) systems [5]. This model, UCSF BERT, was trained on 75 million PHI redacted clinical notes documented across a range of specialties over the last 10 years at the University of California, San Francisco (UCSF). Evaluations of this model on several general benchmarks show that it performs as well as or better than other comparable BERT models not specifically trained from scratch using a diverse corpus of notes derived from EHRs.

### Handling Note Length and Class Imbalance

To include the entire HPI section, which was often longer than the traditional length evaluated by other BERT models, a hierarchical version of the UCSF-BERT model was developed (H-UCSF-BERT) [6]. This model learns to process text using input sequences of 512 tokens (roughly equivalent to words), in the same manner as a typical BERT would. It then combines them into a longer-sequence representation by integrating an additional transformer layer on top of these chunk representations. Sequences up to 2560 tokens were encoded, which is 5 times the usual processing limit of a BERT model. The Mann-Whitney test was utilized to evaluate the relationship of note length and presence of SAE.

SAEs were generally uncommon in the notes corpus, creating a potential problem for training models to learn to positively identify SAEs when they do occur. To optimize learning in the face of this imbalance in the dataset, the training data examples without AEs were randomly undersampled. A range of proportions were explore and a ratio of 1:4 positive to negative examples was settled on as the final choice applied to the training dataset for all downstream tasks. Additional strategies were also explored like weighting the optimization loss based on class distributions, as well as learning these weights dynamically [7]. However, the most promising result was obtained by undersampling the majority dataset.

### Modeling

Several prediction tasks were defined, including classifying whole HPIs according to the occurrence of at least one documented SAE, classifying candidate medication-hospitalization or hospitalization-AE pairs as belonging to a valid SAE triple relationship or not, and classifying candidate medication-hospitalization-AE triples as being a valid SAE (FIG. 1). Models of different architectures were trained to determine which were best suited for the task of AE detection. Automated machine learning was used to train several baseline Bag of Words (BoW) models such as Logistic Regression, K-Nearest Neighbors, Decision Trees, Random Forest and XGBoost. These served as a baseline to compare the performance UCSF-BERT. Deep learning model architectures were adapted such as a Convolutional Neural Network (CNN), Bidirectional a Long Short Term Memory Network (Bi-LSTM12) and a Bi-LSTM with attention. These are deep learning models adapted from the top performing entries in the N2C2 adverse event detection challenge, a nationwide clinical data science competition that was held in 2018.

The baseline model parameters were set to default values and then optimized using grid search. For the deep learning models, hyperparameters were optimized using an incremental approach (e.g. learning rate 0.1, 0.01, 0.001). Hyperparameters in the autoML-trained models were algorithmically optimized. The models were generated in Python 3.8 using Scikit-learn 1.1 for baseline models, deep learning models were generated using PyTorch 1.11.0, and autoML models were generated using AutoGluon 0.4.2 packages respectively.

### Inference Task Set Up

As the first task, a classifier was implemented that takes the HPI section of a note as the input, and outputs whether it contains the mention of a SAE, which was determined as discussed earlier.

In the next set of tasks, relation extraction classifiers were implemented to determine relations between pairs of specific events. Mentions of the individual events of interest were replaced with placeholders, and the model was asked to infer whether a valid relation exists between these placeholders. In the first relation classification task, it was aimed to find whether the patient was on the medication @MED$ before the hospitalization @HOSP$ happened. Here, @MED$ is the placeholder for the medication of interest, and @HOSP$ is the placeholder for a specific hospitalization event.

In the second relation classification task, the model was trained to classify whether a specific adverse event mentioned in the text, represented by the @AE$ token, was the cause of a hospitalization event denoted @HOSP$. These tasks were based on the relations that were added by the annotators while curating the annotated dataset. Better performance may be accomplished by disintegrating the task of SAE detection into these sub-tasks, the need for any causal inferences at this step is alleviated.

In the final task, the model was asked to classify relations between triples of events: a target medication mention, a hospitalization mention, and an adverse event. The relation is present if both the target medication and the adverse event are related to the same hospitalization event, absent otherwise. The UCSF-BERT model was used for performing classification as well as for inferring relations described above.

### Results

### Performance of UCSF-BERT on the Task of SAE Detection

Four targets of prediction were established for all downstream models: 1) identifying notes based on the presence or absence of at least one documented SAE, 2) identifying whether a mentioned medication was documented as being regularly given at the time of a mentioned hospitalization, 3) identifying whether a mentioned diagnosis corresponded to the primary reason for a given hospitalization mention, and 4) identifying whether or not a given triplet of documented medication, hospitalization, and diagnosis corresponded to a valid SAE (FIG. 1). The annotated data was transformed and then split into training, validation, and testing datasets for each of these binary classification tasks (Table 3 and Table 5).

TABLE 5
Positive and Negative Relations for Classification Subtask Triplets

| | Med before hospitalization | | | Hosp for AE | | | Serious AE relations | | |
|---|---|---|---|---|---|---|---|---|---|
| | Train | Dev. | Test | Train | Dev. | Test | Train | Dev. | Test |
| | (%) | (%) | (%) | (%) | (%) | (%) | (%) | (%) | (%) |
| Positive | 1396 | 128 | 168 | 858 | 69 | 95 | 1886 | 209 | 319 |
| relations | (82) | (8) | (10) | (84) | (7) | (9) | (78) | (9) | (13) |
| Negative | 15377 | 1814 | 2162 | 9594 | 1202 | 1119 | 57043 | 6322 | 8409 |
| relations | (80) | (9) | (11) | (81) | (10) | (9) | (80) | (8) | (12) |

Several variations of the UCSF-BERT model were developed and trained to address each of these targets. Their performance against several other comparator models were then evaluated, including several of the top entries from the 2018 N2C2 adverse event detection challenge. All BERT results are from the median performance of Macro F1 score over 5 runs of the model with different seeds. In all the results, negative samples in the training set have been undersampled to the proportion of 80% of positive examples and medication names have been normalized to their generic names.

On the task of medication prior to hospitalization, H-UCSF-BERT was the most performant model with accuracy of 88% and Macro F1 of 0.62 (Table 6). H-UCSF-BERT was more than 10% better compared to the second-best performing model at this task, XGBoost, with an accuracy of 73% and Macro F1 0.51. Similarly, H-UCSF-BERT was the best model at that task of identifying hospitalization relations to SAEs with an accuracy of 96% and Macro F1 of 0.62. It was hypothesized that long-distances between mentions of a hospitalization and the associated SAEs could be reducing performance which prompted trying the model on in the identifying hospitalization relations to SAEs while restricting to SAEs mentioned within 2 sentence spans from the hospitalization. Performance was indeed improved when restricting to SAEs mentioned within a two-sentence span of the hospitalization, Macro F1 increased to 0.68 from 0.62. Whereas the next best performing model at the task of identifying hospitalization relations to SAEs, decision trees, had an accuracy of 86% and Macro F1 0.55. The ultimate goal was to have the model accurately detect triples which include the mention of a steroid-sparing immunosuppressant prior to a hospitalization plus the hospitalization plus the associated SAEs. For the triples task H-UCSF-BERT was again the best performer with an accuracy of 91% and Macro F1 of 0.61. Followed by an auto machine learning model, called NeuralNetFastAI_BAG_L1, which had an accuracy of 84% and Macro F1 of 0.58.

Given the promising performance of H-UCSF-BERT on the aforementioned SAE relational subtasks, the model's performance was tested on whether the note had any SAE mentioned at all. H-UCSF-BERT was able to distinguish if an SAE was present in a document with a Macro F1 of 0.66 (Table 7).

TABLE 6
Performance Results for Three Prediction Tasks

| | | | Macro | | | | |
|---|---|---|---|---|---|---|---|
| Task | Model | Accuracy | F1 | TP | TN | FP | FN |
| Medication | H-UCSF-BERT | 0.88 | 0.62 | 63 | 1989 | 173 | 105 |
| before | Logistic Regression | 0.72 | 0.41 | 42 | 1653 | 509 | 126 |
| hospitalization | XGBoost | 0.73 | 0.51 | 49 | 1672 | 490 | 119 |
| relations | CNN | 0.74 | 0.49 | 51 | 1682 | 480 | 117 |
| | BiLSTM | 0.73 | 0.48 | 51 | 1670 | 492 | 117 |
| Hospitalization | H-UCSF-BERT | 0.96 | 0.62 | 79 | 9603 | 421 | 16 |
| for SAE | H-UCSF-BERT + only | 0.92 | 0.68 | 34 | 1078 | 41 | 61 |
| relations | nearby SAEs | | | | | | |
| | KNNeighbors + only | 0.88 | 0.53 | 11 | 1064 | 55 | 84 |
| | nearby SAEs | | | | | | |
| | Decision Tree + only | 0.86 | 0.55 | 18 | 1038 | 81 | 77 |
| | nearby SAEs | | | | | | |
| | CNN + only nearby AEs | 0.92 | 0.47 | 0 | 1119 | 0 | 95 |
| | BiLSTM + only nearby | 0.93 | 0.48 | 7 | 1091 | 28 | 88 |
| | SAEs | | | | | | |
| | Decision trees (BoW) + | 0.88 | 0.53 | 0 | 8409 | 0 | 319 |
| | only nearby SAEs | | | | | | |
| Medication | H-UCSF-BERT + only | 0.91 | 0.61 | 141 | 7790 | 619 | 178 |
| before | nearby SAEs | | | | | | |
| hospitalization | CNN + only nearby AEs | 0.94 | 0.49 | 11 | 8013 | 396 | 308 |
| for adverse | BiLSTM + only nearby | 0.95 | 0.50 | 11 | 7953 | 456 | 308 |
| event relation | AEs | | | | | | |
| (triples) | BiLSTM + attention + | 0.94 | 0.49 | 0 | 8409 | 0 | 319 |
| | only nearby AEs | | | | | | |
| | AutoML: | 0.84 | 0.58 | 25 | 7362 | 1047 | 294 |
| | NeuralNetFastAI_BAG_L | | | | | | |
| | 1 + only nearby AEs | | | | | | |

TABLE 7
Results of Task Classifying Notes Mentioning
Serious Adverse Drug Events

| Model | Accuracy | Macro F1 | TP | TN | FP | FN |
|---|---|---|---|---|---|---|
| H-UCSF-BERT | 0.67 | 0.66 | 26 | 36 | 18 | 13 |
| CatBoost_BAG_L1 | 0.72 | 0.63 | 36 | 31 | 23 | 3 |

Many aspects make the task of AE detection from clinical notes particularly difficult. These include the length of typical clinical notes, the need to infer relationships between medications and documented AEs, to encode AEs in a standardized way, class imbalance in clinical notes, and to overcome inherent vagueness in the documentation of clinical notes. he present disclosure successfully adapts a new clinical language model, UCSF BERT, to the task of mining for SAE to steroid-sparing immunosuppressants in outpatient clinic notes for patients with IBD. Towards this end a gold standard corpus of nearly 1000 notes was generated as the basis of training and evaluating this model against several baselines. The success of this model appears to stem in part from its pretraining on a large and diverse corpus of notes derived from real-world clinical care and use of hierarchical modeling which allows for long sequence document classification tasks. Inter-rater reliability testing indicates good to excellent concordance across annotators. The best performing model across a range of tasks pertaining to serious AE detection from clinical notes was the H-UCSF-BERT. It achieves macro F1 scores ranging from 62-68% and accuracies from 67-96%, reflecting excellent performance despite significant imbalances in the training data due to the general rarity of SAEs. This model substantially outperforms existing state of the art models for SAE detection associated with the National NLP N2C2 Challenge [9] as well as a range of strong baseline models, including several trained using automated machine learning. As it stands, this pipeline can confidently exclude notes without mentions of serious treatment associated adverse events, substantially reducing the manual efforts required of human reviewers.

The most frequent SAE found in the corpus of outpatient IBD clinical notes at a tertiary referral center was failure of intended efficacy followed by infections. This is in line with previously published data, especially in the setting of >60% of the SAEs in the corpus associated with anti-tumor necrosis factor agents [10]. The corpus includes SAEs from every organ system. Of note, the steroid-sparing medications of interest are not prescribed with equal frequency, thus prescribing practices are likely to influence the frequency of events as well as frequencies of possible AEs inherent to the medication. The text-based automation tool of the present disclosure can be applied to enable more precise characterizations of drug safety in the context of routine clinical care to help validate known safety profiles of these drugs as well as identify previously unrecognized SAEs which can point to areas of inquiry.

### REFERENCES

[1] Thein D, Egeberg A, Skov L, Loft N. Absolute and Relative Risk of New-Onset Psoriasis Associated With Tumor Necrosis Factor-α Inhibitor Treatment in Patients With Immune-Mediated Inflammatory Diseases: A Danish Nationwide Cohort Study. JAMA dermatology. 2022.
[2] Savova G K, Masanz J J, Ogren P V, et al. Mayo clinical Text Analysis and Knowledge Extraction System (cTAKES): architecture, component evaluation and applications. Journal of the American Medical Informatics Association. 2010; 17(5):507-513.
[3] Bodenreider O. The unified medical language system (UMLS): integrating biomedical terminology. Nucleic acids research. 2004; 32(suppl_1):D267-D270.
[4] Label Studio: Data Labeling Software [computer program]. 2020-2022.
[5] Sushil M, Ludwig D, Butte A J, Rudrapatna V A. Developing a general-purpose clinical language inference model from a large corpus of clinical notes. arXiv preprint arXiv:221006566. 2022.
[6] Ji S, Hölttä M, Marttinen P. Does the magic of BERT apply to medical code assignment? A quantitative study. Computers in Biology and Medicine. 2021; 139:104998.
[7] Savova G K, Masanz J J, Ogren P V, et al. Mayo clinical Text Analysis and Knowledge Extraction System (cTAKES): architecture, component evaluation and applications. Journal of the American Medical Informatics Association. 2010; 17(5):507-513.
[8] Bodenreider O. The unified medical language system (UMLS): integrating biomedical terminology. Nucleic acids research. 2004; 32(suppl_1):D267-D270.
[9] Henry S, Buchan K, Filannino M, Stubbs A, Uzuner O. 2018 n2c2 shared task on adverse drug events and medication extraction in electronic health records. Journal of the American Medical Informatics Association. 2019; 27(1):3-12.
[10] Quezada S M, McLean L P, Cross R K. Adverse events in IBD therapy: the 2018 update. Expert Review of Gastroenterology & Hepatology. 2018; 12(12):1183-1191.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims. The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) or 35 U.S.C. § 112(6) is not invoked.

What is claimed is:

1. A method of identifying medical intervention related adverse events, the method comprising:
obtaining a plurality of health records comprising clinical notes regarding individuals associated with the medical intervention;
preprocessing the plurality of clinical notes to include medical intervention and adverse events labels;
annotating a first subset of the preprocessed clinical notes to indicate if each labeled adverse event is associated with the medical intervention;
training a machine learning model to identify adverse events associated with the medical intervention using the annotated clinical notes;
applying the trained machine learning model to a second subset of the preprocessed clinical notes to identify adverse events associated with the medical intervention.

2. The method according to claim 1, wherein the method further comprises training the machine learning model with a first subset of the annotated clinical notes and testing the trained machine learning model using a second subset of the annotated clinical notes.

3. The method according to claim 2, wherein the method further comprises validating the trained machine learning model using a third subset of the annotated clinical notes.

4. The method according to any of the preceding claims, wherein the method further comprises normalizing the wording of the identified adverse events associated with the medical intervention.

5. The method according to claim 4, wherein the method further comprises transmitting the normalized adverse events.

6. The method according to any of the preceding claims, wherein the preprocessing further comprises reducing the plurality of clinical notes to only the most recent note of each individual.

7. The method according to claim 6, wherein the plurality of clinical notes is reduced to only the history of present illness section of the notes.

8. The method according to any of the preceding claims, wherein the preprocessing further comprises labeling hospitalizations.

9. The method according to any of the preceding claims, wherein the labeling is performed automatically.

10. The method according to claim 9, wherein the automatic labelling is performed using a clinical natural language processing software.

11. The method according to claim 10, wherein the clinical natural language processing software is the clinical Text Analysis and Knowledge Extraction System (cTAKES).

12. The method according to any of the preceding claims, wherein the preprocessing further comprises normalizing the wording of the labeled medical intervention.

13. The method according to claim 12, wherein the normalizing is performed using the Unified Medical Language System (UMLS).

14. The method according to claim 8, wherein the annotating further comprises indicating if the medical intervention occurred prior to hospitalization.

15. The method according to any of the preceding claims, wherein the annotating is performed by a medical professional or a pharmacovigilance specialist.

16. The method according to any of the preceding claims, wherein the annotating is performed by more than one individual.

17. The method according to claim 15 or 16, wherein an interrater reliability assessment is performed on the annotations provided from different annotators.

18. The method according to claim 17, wherein the assessment is a Fleiss' kappa statistic.

19. The method according to claim 17 or 18, wherein the assessment is used to determine if the annotating step is adjusted and reperformed.

20. The method according to any of the preceding claims, wherein the machine learning model is a deep learning model.

21. The method according to claim 20, wherein the machine learning model is 12 or more layers deep.

22. The method according to any of the preceding claims, wherein the machine learning model is a transformer.

23. The method according to claim 22, wherein the machine learning model comprises encoder layers.

24. The method according to any of the preceding claims, wherein the machine learning model is a masked-language model.

25. The method according to claim 24, wherein the masked-language model is a Bidirectional Encoder Representations from Transformers (BERT) model.

26. The method according to any of the preceding claims, wherein the machine learning model comprises a hierarchical architecture.

27. The method according to any of the preceding claims, wherein the machine learning model is pretrained to interpret clinical text.

28. The method according to any of the preceding claims, wherein the method further comprises pretraining using publicly available adverse event data.

29. The method according to claim 28, wherein the adverse event data is data from a clinical trial.

30. The method according to any of the preceding claims, wherein the annotated clinical notes without labeled adverse events are randomly undersampled during training.

31. The method according to claim 30, wherein the undersampling results in the number of clinical notes without labeled adverse events being less than five times greater than the number of clinical notes having labeled adverse events.

32. The method according to claim 2, wherein the testing is used to determine if the trained machine learning model meets a predetermined threshold of accuracy.

33. The method according to claim 32, wherein the trained machine learning model is discarded if the predetermined threshold of accuracy is not met.

34. The method according to claim 3, wherein a plurality of machine learning models are trained concurrently and the validating is used to select one of the trained machine learning models for testing.

35. The method according to any of the preceding claims, wherein the medical intervention is the use of a medical device, undergoing a surgery, receiving a medication, or undergoing a therapy.

36. The method according to claim 35, wherein the medical intervention is the use of a medical device or receiving a medication.

37. The method according to claim 35, wherein the medical intervention is the receiving of a medication.

38. The method according to any of the preceding claims, wherein the method further comprises identifying adverse events related to a plurality of medical interventions.

39. The method according to claim 35, wherein the plurality of medical interventions is all associated with the same condition or disease.

40. The method according to any of the preceding claims, wherein the adverse events include one or more of infections and infestations, lack of intended medical intervention efficacy, gastrointestinal disorders, general disorders and administration site conditions, blood and lymphatic system disorders, psychiatric disorders, endocrine disorders, cardiac disorders, respiratory, thoracic and mediastinal disorders, renal and urinary disorders, nervous system disorders, eye disorders, inflammation, hepatobiliary disorders, injury, poisoning and procedural complications, metabolism and nutrition disorders, vascular disorders, neoplasms, and/or skin and subcutaneous tissue disorders

41. The method according to any of the preceding claims, wherein the method further comprises training the machine learning model to exclude clinical notes that do not include medical intervention associated adverse events.

42. The method according to claim 41, wherein the method further comprises applying the trained machine learning model to exclude clinical notes that do not include medical intervention associated adverse events.

43. The method according to claim 8, wherein the medical intervention is associated with a disease or condition and the method further comprises training the machine learning model to determine if hospitalizations were associated with the disease or condition.

44. The method according to claim 43, wherein the method further comprises applying the trained machine learning model to determine if hospitalizations were associated with the disease or condition.

45. The method according to claim 8, wherein the medical intervention is associated with a disease or condition diagnosed in the individuals and the method further comprises training the machine learning model to identify if triplets of medical intervention, hospitalization, and diagnosis of the disease or condition indicating valid medical intervention associated adverse events.

46. The method according to claim 45, wherein the method further comprises applying the trained machine learning model to identify medical intervention associated adverse events validated by triplets of medical intervention, hospitalization, and diagnosis of the disease or condition.

47. A system for identifying medical intervention related adverse events, the system comprising:
a processor comprising memory operably coupled to the processor, wherein the memory comprises instructions stored thereon, which, when executed by the processor, cause the processor to:
obtain a plurality of health records comprising clinical notes regarding individuals associated with the medical intervention;
preprocess the plurality of clinical notes to include medical intervention and adverse event labels;
annotate a first subset of the preprocessed clinical notes to indicate if each labeled adverse event is associated with the medical intervention;
train a machine learning model to identify adverse events associated with the medical intervention using the annotated first subset of clinical notes;
apply the trained machine learning model to a second subset of the preprocessed clinical notes to identify adverse events associated with the medical intervention.

48. The system according to claim 47, wherein the memory comprises instructions stored thereon, which when executed by the processor, cause the processor to train the machine learning model with a first subset of the annotated clinical notes and test the trained machine learning model using a second subset of the annotated clinical notes.

49. The system according to claim 48, wherein the memory comprises instructions stored thereon, which when executed by the processor, cause the processor to validating the trained machine learning model using a third subset of the annotated clinical notes.

50. The system according to any of claims 47 to 49, wherein the memory comprises instructions stored thereon, which when executed by the processor, cause the processor to normalize the wording of the identified adverse effects associated with the medical intervention.

51. The system according to any of claims 47 to 50, wherein the memory comprises instructions stored thereon, which when executed by the processor, cause the processor to transmit the normalized adverse effects.

52. The system according to any of claims 47 to 51, wherein the preprocessing further comprises reducing the plurality of clinical notes to only the most recent note of each individual.

53. The system according to claim 52, wherein the plurality of clinical notes is reduced to only the history of present illness section of the notes.

54. The system according to any of claims 47 to 53, wherein the preprocessing is performed using a clinical natural language processing software.

55. The system according to claim 54, wherein the clinical natural language processing software is the clinical Text Analysis and Knowledge Extraction System (cTAKES).

56. The system according to any of claims 47 to 55, wherein the memory comprises instructions stored thereon, which when executed by the processor, cause the processor to normalize the wording of the labeled medical intervention.

57. The system according to claim 56, wherein the generalizing is performed using the Unified Medical Language System (UMLS).

58. The system according to any of claims 47 to 57, wherein the annotating is based on the input provided by one or more annotators.

59. The system according to claim 58, wherein the system further comprises input means configured to receive input provided by one or more annotators and transmit it to the processor.

60. The system according to claim 58, wherein the memory comprises instructions stored thereon, which when executed by the processor, cause the processor to instruct the one or more annotators to provide annotation input.

61. The system according to claim 58, wherein the system further comprises a visual display configured to instruct the one or more annotators to provide annotation input.

62. A computer program product embodied on a non-transitory computer readable medium, the computer program product comprising:
code for obtaining a plurality of health records comprising clinical notes regarding individuals associated with the medical intervention;
code for preprocessing the plurality of clinical notes to include medical intervention and adverse event labels;
code for annotating a first subset of the preprocessed clinical notes to indicate if each labeled adverse event is associated with the medical intervention;
code for training a machine learning model to identify adverse events associated with the medical intervention using the annotated first subset of clinical notes;
code for applying the trained machine learning model to a second subset of the preprocessed clinical notes to identify adverse events associated with the medical intervention.

63. The product according to claim 62, wherein the product further comprises code for training the machine learning model with a first subset of the annotated clinical notes and testing the trained machine learning model using a second subset of the annotated clinical notes.

64. The product according to claim 62, wherein the product further comprises code for validating the trained machine learning model using a third subset of the annotated clinical notes.

65. The product according to any of claims 62 to 64, wherein the product further comprises code for normalizing the wording of the identified adverse events associated with the medical intervention.

66. The product according to claim 65, wherein the product further comprises code for transmitting the normalized adverse events.

67. The method according to any of claims 62 to 66, wherein the preprocessing further comprises code for reducing the plurality of clinical notes to only the most recent note of each individual.

68. The product according to claim 67, wherein the plurality of clinical notes is reduced to only the history of present illness section of the notes.

69. The product according to any of claims 62 to 68, wherein the preprocessing further comprises code for labeling hospitalizations.

70. The product according to claim 69, wherein the labelling is performed using a clinical natural language processing software.

71. The product according to claim 70, wherein the clinical natural language processing software is the clinical Text Analysis and Knowledge Extraction System (cTAKES).

72. The product according to any of claims 62 to 71, wherein the preprocessing further comprises code for normalizing the wording of the labeled medical intervention.

73. The product according to claim 72, wherein the normalizing is performed using the Unified Medical Language System (UMLS).
